(19) United States
(12) Patent Application Publication
(10) Pub. No.: US 2026/0355261 A1
(43) Pub. Date: Nov. 12, 2026

(54) CORYNEBACTERIUM STRAINS, COMBINATIONS, AND LYOPHILIZED FORMULATIONS THEREOF FOR USE IN THE PREVENTION OF A VIRAL INFECTION

(71) Applicant: SEMMELWEIS EGYETEM, Budapest (HU)

(72) Inventors: Dóra SZABÓ, Budapest (HU); László TAMÁS, Budapest (HU); Eszter OSTORHÁZI, Diósd (HU); István ANTAL, Budapest (HU); Balázs STERCZ, Kakasd (HU); Emese BERTALANNÉ BALOGH, Budapest (HU); Béla MERKELY, Budapest (HU)

(21) Appl. No.: 19/120,855

(22) PCT Filed: Oct. 12, 2023
(86) PCT No.: PCT/HU2023/050072

§ 371 Date: Apr. 14, 2025

(30) Foreign Application Priority Data
Oct. 12, 2022 (HU) P2200412

(51) Int. Cl. A61K 35/741; A61K 9/00; A61K 9/19; A61K 47/36; A61P 31/14

(52) U.S. Cl. CPC A61K 35/741; A61K 9/0043; A61K 9/0073; A61K 9/19; A61K 47/36; A61P 31/14

(57) ABSTRACT

The invention relates to Corynebacterium strains capable of reducing the expression of cathepsin, and their combinations for use in the prevention of a viral infection, in particular SARS-CoV-2 infection, or for use in the prevention of an infection by an enveloped respiratory virus in a subject, wherein said virus uses cathepsin entry route for entry into the cell of the subject. In particular, said virus is SARS-CoV-2 omicron variant. The invention also relates to lyophilized formulations comprising Corynebacterium strains or combinations thereof, and a cryoprotectant, for use in the prevention of an infection by an enveloped respiratory virus, preferably a SARS-CoV-2 virus in a subject. Preferably, the formulation is administered to the upper respiratory tract of the subject.

### FIELD OF THE INVENTION

The invention relates to Corynebacterium strains capable of reducing the expression of cathepsin, and their combinations for use in the prevention of a viral infection, in particular SARS-CoV-2 infection. The invention also relates to lyophilized formulations comprising these Corynebacterium strains or combinations, for use in the prevention of coronavirus infection.

### BACKGROUND ART

The epithelial cells of the upper and lower respiratory tracts are the primary targets for airborne infections. These cells are covered by complex bacterial communities mainly in the upper respiratory tract that may directly or indirectly interact with coronaviruses. The idea that commensal bacteria may also prevent infection by regulating innate and adaptive host immune responses has already been raised in the art. Furthermore, nasopharyngeal or oronasopharyngeal preparations comprising bacteria to alter the composition of the microbiome in the upper respiratory tract have been proposed.

Lappan and Peacock [Lappan & Peacock, 2019. Corynebacterium and Dolosigranulum: future probiotic candidates for upper respiratory tract infections. Microbiology Australia, 40(4), 172-177.] review studies observing the presence of Corynebacterium and Dolosigranulum in the microbiome of the upper respiratory tract and which suggest their association with a healthy state. The authors advise that commensal Corynebacterium pseudodiphtheriticum and Dolosigranulum pigrum strains, which are held to be pathogens of the upper respiratory tract, may have protective role in the nasopharyngeal health of children.

Man et al. in their paper published in 2017 also came to the conclusion that Dolosigranulum spp. and Corynebacterium spp. are potential keynote players in the upper respiratory tract microbiota as “they have been strongly associated with respiratory health and the exclusion of potential pathogens, most notably Streptococcus pneumoniae, in several epidemiological and mechanistic studies”. [Man et al., 2017. The microbiota of the respiratory tract: Gatekeeper to respiratory health. Nature Reviews Microbiology, 15(5), 259-270.]

The question of the interrelations of the healthy state of the microbiome and serious infections by RNA viruses, among other coronavirus infections have also been raised recently in several studies.

The highly transmissible and pathogenic coronavirus that emerged in late 2019, i.e., severe acute respiratory syndrome coronavirus 2 (SARS-CoV-2) resulting in coronavirus disease 2019 (COVID-19) has caused a pandemic. The importance of this research is more than supported by the fact that by Sep. 21, 2022, a total of 609 million diagnosed cases and 6.5 million deaths due to COVID-19 have been confirmed by WHO (https://www.who.int/publications/m/item/weekly-epidemiological-update-on-covid-19-21-september-2022).

SARS-CoV-2 infection seems to have a dual nature: tragically lethal in some persons, surprisingly mild in others, and there were even people who were not susceptible to the virus at all.

Infection caused by the SARS-CoV-2 virus is a serious problem. The virus settles primarily on the nasal mucosa, then the infection develops into a severe systemic disease. The infection and its course depend on individual susceptibility.

The epithelial cells of the upper and lower respiratory tracts are the primary targets for SARS-CoV-2 virus infection and replication. Assuming that the intrusion gate is guarded by a commensal microbiome of the nasopharynx, several authors have tested the bacterial components in infected and non-infected individuals.

Nardelli et al. [Nardelli et al., 2021. Nasopharyngeal Microbiome Signature in COVID-19 Positive Patients: Can We Definitively Get a Role to Fusobacterium periodonticum? Frontiers in Cellular and Infection Microbiology, 11(February), 1-7.] and Maio et al. [Maio et al., 2020. Nasopharyngeal Microbiota Profiling of SARS-CoV-2 Infected Patients. Biological Procedures Online 22, 18.] found no difference in nasopharyngeal microbiome composition in the samples from COVID-19 patients and virus-negative controls. Rather, it can be concluded from their data that SARS-CoV-2 infection did not significantly alter the composition of the microbiome compared to the microbiome of the average population. Testing larger number of patients and using the 16S rRNA method, Rosas-Salazar et al. [Rosas-Salazar et al., 2021. SARS-CoV-2 infection and viral load are associated with the upper respiratory tract microbiome. J Allergy Clin Immunol. 2021 April; 147(4):1226-1233.e2] reported a mixed picture of Corynebacteria: while some strains were more abundant in SARS-CoV-2 infected subjects, others were more abundant in those not infected with the virus.

Metagenomic next-generation sequencing in contrast to targeted methods also provides valuable information on the composition of the microbiome at the species level. The metagenomic next-generation study of Mostafa et al. [Mostafa et al., 2020. Metagenomic next-generation sequencing of nasopharyngeal specimens collected from confirmed and suspect COVID-19 patients. MBio, 11(6), 1-13.] described a statistically significant decrease of incidence of a commensal organism, Corynebacterium accolens in the samples of COVID-19-positive patients. The authors mention that there is evidence in the prior art that C. accolens has a negative association with colonization by Streptococcus pneumoniae; emphasize, however, that further studies would be required to conclude the role of these associations in patients with COVID-19 and do not raise that there may be a negative association between the presence of C. accolens and SARS-CoV-2 infection. Actually, the decrease of the level of C. accolens may well be a result of the infection.

Susceptibility to the SARS-CoV-2 infection and its clinical course is still unpredictable. Tchoupou Saha et al. [Tchoupou Saha et al., 2022. Profile of the Nasopharyngeal Microbiota Affecting the Clinical Course in COVID-19 Patients. Front. Microbiol. 13:871627.] studied nasopharyngeal microbiota of COVID-19 patients and patients tested negative for the virus, by 16S ribosomal ribonucleic acid (rRNA) sequencing and specific polymerase chain reaction (PCR) targeting pathogens. They found that in patients tested positive for SARS-CoV-2, 9 taxa were increased, e.g., Corynebacterium propinquum/pseudodiphtericum and Afipia birgiae. They also demonstrated that C. propinquum is decreased in asymptotic individuals compared to other COVID-19 positive patients. However, these authors also state that further studies are needed to determine the exact role in the clinical course of the disease of Corynebacterium accolens, and more specifically Corynebacterium propinquum/diphteriticum.

The COVID-19 epidemic is still a serious problem. One of the reasons for this is that the evolution of SARS-CoV-2 has led to the emergence of several new variants. The newly released SARS-CoV-2 omicron variant has become the dominant circulating variant in many countries. Due to the large number of mutations, omicron shows cell tropism and mode of entry compared to other SARS-CoV-2 variants. Unlike the original or other SARS-CoV-2 variants, the omicron variant primarily uses the Cathepsin B/L entry route in addition to the TMPRSS2 (transmembrane protease/serine subfamily member 2) cell entry route.

The present inventors' aims were to isolate bacterium strains downregulating the Cathepsin pathway that can be used as probiotics in the prevention of respiratory viral infections, particularly SARS-CoV-2 infection; to determine ideal combinations of these strains; and to formulate nasal sprays (lyophilized formulations) comprising these strains or combinations together with appropriate excipients.

### BRIEF DESCRIPTION OF THE INVENTION

In a first aspect, a Corynebacterium culture is provided for use in the prevention or the treatment of an infection by an enveloped respiratory virus, preferably a coronavirus in a subject, wherein the Corynebacterium culture is capable of reducing the expression of a protein facilitating entry of the enveloped respiratory virus into a cell of the subject, wherein said entry facilitating protein is a cathepsin. Preferably, a Corynebacterium culture is provided for use in the prevention or the treatment of an infection by an enveloped respiratory virus, preferably a coronavirus in a subject, wherein the Corynebacterium culture is capable of reducing the expression of a cathepsin in a cell of the subject. Optionally, said Corynebacterium culture is also capable of reducing the expression of further proteins facilitating entry of the enveloped respiratory virus, preferably coronavirus, into a cell of the subject, selected from angiotensin-converting enzyme 2 (ACE2) and transmembrane protease/serine subfamily member 2 (TMPRSS2).

In an embodiment, a Corynebacterium culture is provided for use in the prevention or the treatment of an infection by an enveloped respiratory virus, preferably a coronavirus in a subject, wherein the Corynebacterium culture is capable of reducing the expression of at least one protein facilitating entry of the enveloped respiratory virus into a cell of the subject, wherein said at least one entry facilitating protein is selected from cathepsin, ACE2 and TMPRSS2, or any combinations thereof, wherein preferably said entry facilitating protein is cathepsin, or wherein preferably said entry facilitating proteins are cathepsin and ACE2, or wherein said entry facilitating proteins are cathepsin, ACE2 and TMPRSS2.

Preferably, the enveloped respiratory virus is a coronavirus, preferably SARS-CoV-2 and more preferably SARS-CoV-2 omicron variant.

In a second aspect, a formulation is provided for use in the prevention or the treatment of an infection by a coronavirus in a subject, wherein the formulation comprises a Corynebacterium culture capable of reducing expression of a protein facilitating entry of the coronavirus into a cell of the subject, said entry facilitating protein being a cathepsin, and wherein optionally said Corynebacterium culture may also be capable of reducing the expression of further proteins facilitating entry of the coronavirus into a cell of the subject, selected from ACE2 and TMPRSS2. Preferably the formulation is a probiotic composition. Preferably the formulation is (part of) a medical device. Preferably, the formulation is delivered by a medical device. Preferably the formulation is a pharmaceutical formulation. Preferably the formulation also comprises at least one excipient suitable for a probiotic formulation and/or a pharmaceutically acceptable excipient, when appropriate.

In a third aspect, a composition is provided, comprising a Corynebacterium culture capable of reducing expression of a protein facilitating entry of a coronavirus into a cell of a subject, said entry facilitating protein being a cathepsin, and wherein optionally said Corynebacterium culture may also be capable of reducing the expression of further proteins facilitating entry of the coronavirus into a cell of the subject, selected from ACE2 and TMPRSS2. Preferably the composition is a probiotic composition. Preferably the composition is (part of) a medical device. Preferably, the composition is delivered by a medical device. Preferably the composition is a pharmaceutical composition. Preferably the composition is for use in the prevention or treatment of a coronavirus infection. Preferably the composition also comprises at least one excipient suitable for a probiotic composition and/or a pharmaceutically acceptable excipient, when appropriate.

In a fourth aspect a method is provided for the prevention or the treatment of an infection by a coronavirus, the method comprising administering a Corynebacterium culture or a formulation or composition comprising a Corynebacterium culture to a subject in need thereof, wherein the Corynebacterium culture is capable of reducing expression of a protein facilitating entry of the coronavirus into a cell of the subject, said entry facilitating protein being a cathepsin, and wherein optionally said Corynebacterium culture may also be capable of reducing the expression of further proteins facilitating entry of the coronavirus into a cell of the subject, selected from ACE2 and TMPRSS2.

In any one of the aspects of the invention, preferably the Corynebacterium culture is for use in the prevention of an infection by a coronavirus and thereby also for the prevention of a disease caused by the coronavirus.

In any one of the aspects of the invention, preferably the Corynebacterium culture is also capable of inhibiting binding between the Spike (S) protein of the coronavirus, preferably the receptor binding domain (RBD) of the S protein, and ACE2.

In any one of the aspects of the invention, preferably the Corynebacterium culture comprises viable Corynebacteria selected from the group of strains of Corynebacterium accolens, Corynebacterium propinquum and Corynebacterium tuberculostearicum. Preferably, the Corynebacterium culture comprises strains of Corynebacterium accolens, Corynebacterium propinquum and Corynebacterium tuberculostearicum. Preferably, the Corynebacterium culture comprises at least one strain of Corynebacterium accolens. Preferably, the Corynebacterium culture comprises at least one strain of Corynebacterium propinquum. Preferably, the Corynebacterium culture comprises at least one strain of Corynebacterium tuberculostearicum.

In any one of the aspects of the invention, preferably the Corynebacterium culture does not comprise Corynebacterium pseudodiphtheriticum.

In any one of the aspects of the invention, preferably the Corynebacterium culture comprises at least one Corynebacterium strain selected from:
Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding, and/or is expressing LipS1 lipase and/or is comprising a sequence coding for LipS1 lipase;
Corynebacterium propinquum SU002 strain having the accession number NCAIM P (B) 001496 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding;
Corynebacterium propinquum SU003 strain having the accession number NCAIM P (B) 001497 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding;
Corynebacterium accolens SU004 strain having the accession number NCAIM P (B) 001500 deposited at NCAIM on Jan. 17, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding.

In any one of the aspects of the invention, preferably the Corynebacterium culture comprises at least one Corynebacterium strain selected from:
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jan. 17, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding.

In any one of the aspects of the invention, preferably the Corynebacterium culture comprises at least one Corynebacterium strain selected from:
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jan. 17, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding.

In any one of the aspects of the invention, preferably the Corynebacterium culture comprises at least one Corynebacterium strain selected from:
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jan. 17, 2022, or a derivative, variant or mutant thereof, wherein the derivative, variant or mutant thereof is capable of reducing cathepsin expression;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression;
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression.

In another aspect, the invention relates to Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding, and/or is expressing LipS1 lipase and/or is comprising a sequence coding for LipS1 lipase. The invention also relates to a Corynebacterium culture comprising or essentially consisting of Corynebacterium accolens SU001. Preferably, the Corynebacterium accolens SU001 strain or the culture comprising or essentially consisting of Corynebacterium accolens SU001 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium propinquum SU002 strain having the accession number NCAIM P (B) 001496 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding. The invention also relates to a Corynebacterium culture comprising or essentially consisting of Corynebacterium propinquum SU002. Preferably, the Corynebacterium propinquum SU002 strain or the culture comprising or essentially consisting of Corynebacterium propinquum SU002 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium propinquum SU003 strain having the accession number NCAIM P (B) 001497 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding. The invention also relates to a Corynebacterium culture comprising or essentially consisting of Corynebacterium propinquum SU003. Preferably, the Corynebacterium propinquum SU003 strain or the culture comprising or essentially consisting of Corynebacterium propinquum SU003 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium accolens SU004 strain having the accession number NCAIM P (B) 001500 deposited at NCAIM on Jan. 17, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme. The invention also relates to a Corynebacterium culture comprising or essentially consisting of Corynebacterium accolens SU004. Preferably, the Corynebacterium accolens SU004 strain or the culture comprising or essentially consisting of Corynebacterium accolens SU004 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding. The invention also relates to a Corynebacterium culture comprising or essentially consisting of Corynebacterium tuberculostearicum SU005. Preferably, the Corynebacterium tuberculostearicum SU005 strain or the culture comprising or essentially consisting of Corynebacterium tuberculostearicum SU005 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme. The invention also relates to a Corynebacterium culture comprising or essentially consisting of Corynebacterium accolens SU006. Preferably, the Corynebacterium accolens SU006 strain or the culture comprising or essentially consisting of Corynebacterium accolens SU006 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding. The invention also relates to a Corynebacterium culture comprising or essentially consisting of Corynebacterium propinquum SU007. Preferably, the Corynebacterium propinquum SU007 strain or the culture comprising or essentially consisting of Corynebacterium propinquum SU007 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme. The invention also relates to a Corynebacterium culture comprising or essentially consisting of Corynebacterium accolens SU008. Preferably, the Corynebacterium accolens SU008 strain or the culture comprising or essentially consisting of Corynebacterium accolens SU008 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme. The invention also relates to a Corynebacterium culture comprising or essentially consisting of Corynebacterium accolens SU009. Preferably, the Corynebacterium accolens SU009 strain or the culture comprising or essentially consisting of Corynebacterium accolens SU009 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding. The invention also relates to a Corynebacterium culture comprising or essentially consisting of Corynebacterium propinquum SU010. Preferably, the Corynebacterium propinquum SU010 strain or the culture comprising or essentially consisting of Corynebacterium propinquum SU010 strain is for use in the prevention of an infection by an enveloped respiratory virus, preferably a coronavirus, more preferably a SARS-CoV-2 virus, in a subject. In an embodiment, the SARS-CoV-2 virus is SARS-CoV-2 omicron variant.

In another aspect, the invention relates to a Corynebacterium strain selected from the group consisting of:
Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding, and/or is expressing LipS1 lipase and/or is comprising a sequence coding for LipS1 lipase;
Corynebacterium propinquum SU002 strain having the accession number NCAIM P (B) 001496 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding;
Corynebacterium propinquum SU003 strain having the accession number NCAIM P (B) 001497 deposited at NCAIM on Jun. 22, 2021, or a derivative, variant or mutant thereof, wherein the derivative, variant or mutant thereof is capable of reducing cathepsin expression, and optionally, the derivative, variant or mutant thereof is also capable of reducing ACE2 expression and/or TMPRSS2 expression and/or inhibiting ACE2 and S-protein binding;
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jan. 17, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding and/or is expressing lipase enzyme and/or comprises a sequence coding for lipase enzyme; and
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and optionally, the derivative, variant or mutant thereof is also capable of reducing ACE2 expression and/or reducing TMPRSS2 expression and/or inhibiting ACE2 and S-protein binding.

Preferably, the Corynebacterium culture consists essentially of Corynebacterium, preferably essentially of Corynebacterium accolens and/or Corynebacterium propinquum and/or Corynebacterium tuberculostearicum.

In particular, the Corynebacterium culture comprises a viable Corynebacterium strain in a medium.

In a preferred embodiment of the invention, the Corynebacterium culture comprises a viable Corynebacterium strain selected from the group of strains of Corynebacterium accolens, Corynebacterium propinquum, and Corynebacterium tuberculostearicum.

In any one of the aspects of the invention, preferably the Corynebacterium culture comprises at least two Corynebacterium strains selected from Corynebacterium strains SU001 to SU010 or any derivative, variant or mutant thereof capable of reducing cathepsin expression described above. The Corynebacterium cultures comprising at least two different Corynebacterium strains are also called combinations.

Preferably, the combination comprises at least two viable Corynebacterium strains selected from the group of strains of Corynebacterium accolens, Corynebacterium propinquum and Corynebacterium tuberculostearicum.

Preferably, the combination comprises at least two strains, wherein said strains belong to the same species selected from the group consisting of Corynebacterium accolens, Corynebacterium propinquum and Corynebacterium tuberculostearicum. Preferably, the combination comprises at least two strains, wherein at least two strains belong to different species selected from the group consisting of Corynebacterium accolens, Corynebacterium propinquum and Corynebacterium tuberculostearicum. Preferably, the combination comprises at least two strains selected from the group consisting of Corynebacterium accolens SU001, Corynebacterium propinquum SU002, Corynebacterium propinquum SU003, Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression. Preferably, the combination comprises at least two strains selected from the group consisting of Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression.

Preferably, the combination comprises at least three viable Corynebacterium strains selected from the group of strains of Corynebacterium accolens, Corynebacterium propinquum and Corynebacterium tuberculostearicum. Preferably, the combination comprises at least three strains, wherein at least two strains belong to different species selected from the group consisting of Corynebacterium accolens, Corynebacterium propinquum and Corynebacterium tuberculostearicum. Preferably, the combination comprises at least three strains selected from the group consisting of Corynebacterium accolens SU001, Corynebacterium propinquum SU002, Corynebacterium propinquum SU003, Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression. Preferably, the combination comprises at least three strains selected from the group consisting of Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression.

Preferably, the combination comprises four viable Corynebacterium strains selected from the group of strains of Corynebacterium accolens, Corynebacterium propinquum and Corynebacterium tuberculostearicum. Preferably, the combination comprises four strains, wherein at least two strains belong to different species selected from the group consisting of Corynebacterium accolens, Corynebacterium propinquum and Corynebacterium tuberculostearicum. Preferably, the combination comprises four strains, wherein at least one strain is of Corynebacterium accolens, at least one strain is of Corynebacterium propinquum and at least one strain is of Corynebacterium tuberculostearicum. Preferably, the combination comprises four strains selected from the group consisting of Corynebacterium accolens SU001, Corynebacterium propinquum SU002, Corynebacterium propinquum SU003, Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression. Preferably, the combination comprises four strains selected from the group consisting of Corynebacterium accolens SU004, Corynebacterium tuberculostearicum SU005, Corynebacterium accolens SU006, Corynebacterium propinquum SU007, Corynebacterium accolens SU008, Corynebacterium accolens SU009, Corynebacterium propinquum SU010, and any derivative, variant or mutant thereof capable of reducing cathepsin expression.

In a preferred embodiment, the combination comprises at least two strains, wherein the at least two strains are Corynebacterium accolens SU004 and Corynebacterium tuberculostearicum SU005, or any derivative, variant or mutant thereof capable of reducing cathepsin expression. In another preferred embodiment, the combination comprises at least two strains, wherein the at least two strains are Corynebacterium accolens SU004 and Corynebacterium accolens SU009, or any derivative, variant or mutant thereof capable of reducing cathepsin expression. In another preferred embodiment, the combination comprises at least two strains, wherein the at least two strains are Corynebacterium accolens SU004 and Corynebacterium propinquum SU010, or any derivative, variant or mutant thereof capable of reducing cathepsin expression. In another preferred embodiment, the combination comprises at least two strains, wherein the at least two strains are Corynebacterium tuberculostearicum SU005 and Corynebacterium accolens SU009, or any derivative, variant or mutant thereof capable of reducing cathepsin expression. In another preferred embodiment, the combination comprises at least two strains, wherein the at least two strains are Corynebacterium tuberculostearicum SU005 and Corynebacterium propinquum SU010, or any derivative, variant or mutant thereof capable of reducing cathepsin expression. In another preferred embodiment, the combination comprises at least two strains, wherein the at least two strains are Corynebacterium accolens SU009 and Corynebacterium propinquum SU010, or any derivative, variant or mutant thereof capable of reducing cathepsin expression. In another preferred embodiment, the combination comprises at least two strains, wherein the at least two strains are Corynebacterium accolens SU004 and Corynebacterium accolens SU006, or any derivative, variant or mutant thereof capable of reducing cathepsin expression. In another preferred embodiment, the combination comprises at least two strains, wherein the at least two strains are Corynebacterium tuberculostearicum SU005 and Corynebacterium accolens SU006, or any derivative, variant or mutant thereof capable of reducing cathepsin expression. In another preferred embodiment, the combination comprises at least two strains, wherein the at least two strains are Corynebacterium accolens SU006 and Corynebacterium propinquum SU010, or any derivative, variant or mutant thereof capable of reducing cathepsin expression. In another preferred embodiment, the combination comprises at least two strains, wherein the at least two strains are Corynebacterium accolens SU004 and Corynebacterium propinquum SU007, or any derivative, variant or mutant thereof capable of reducing cathepsin expression. In another preferred embodiment, the combination comprises at least two strains, wherein the at least two strains are Corynebacterium accolens SU004 and Corynebacterium accolens SU008, or any derivative, variant or mutant thereof capable of reducing cathepsin expression. In another preferred embodiment, the combination comprises at least two strains, wherein the at least two strains are Corynebacterium tuberculostearicum SU005 and Corynebacterium propinquum SU007, or any derivative, variant or mutant thereof capable of reducing cathepsin expression. In another preferred embodiment, the combination comprises at least two strains, wherein the at least two strains are Corynebacterium tuberculostearicum SU005 and Corynebacterium accolens SU008, or any derivative, variant or mutant thereof capable of reducing cathepsin expression. In another preferred embodiment, the combination comprises at least two strains, wherein the at least two strains are Corynebacterium propinquum SU007 and Corynebacterium accolens SU008, or any derivative, variant or mutant thereof capable of reducing cathepsin expression. In another preferred embodiment, the combination comprises at least two strains, wherein the at least two strains are Corynebacterium accolens SU006 and Corynebacterium propinquum SU007, or any derivative, variant or mutant thereof capable of reducing cathepsin expression. In another preferred embodiment, the combination comprises at least two strains, wherein the at least two strains are Corynebacterium accolens SU006 and Corynebacterium accolens SU008, or any derivative, variant or mutant thereof capable of reducing cathepsin expression. In another preferred embodiment, the combination comprises at least two strains, wherein the at least two strains are Corynebacterium accolens SU006 and Corynebacterium accolens SU009, or any derivative, variant or mutant thereof capable of reducing cathepsin expression. In another preferred embodiment, the combination comprises at least two strains, wherein the at least two strains are Corynebacterium propinquum SU007 and Corynebacterium accolens SU009, or any derivative, variant or mutant thereof capable of reducing cathepsin expression. In another preferred embodiment, the combination comprises at least two strains, wherein the at least two strains are Corynebacterium propinquum SU007 and Corynebacterium propinquum SU010, or any derivative, variant or mutant thereof capable of reducing cathepsin expression. In another preferred embodiment, the combination comprises at least two strains, wherein the at least two strains are Corynebacterium accolens SU008 and Corynebacterium accolens SU009, or any derivative, variant or mutant thereof capable of reducing cathepsin expression. In another preferred embodiment, the combination comprises at least two strains, wherein the at least two strains are Corynebacterium accolens SU008 and Corynebacterium propinquum SU010, or any derivative, variant or mutant thereof capable of reducing cathepsin expression.

In a preferred embodiment, the combination comprises at least three strains, wherein the at least three strains are Corynebacterium accolens SU004, Corynebacterium tuberculostearicum SU005 and Corynebacterium accolens SU009, or any derivative, variant or mutant thereof capable of reducing cathepsin expression. In another preferred embodiment, the combination comprises at least three strains, wherein the at least three strains are Corynebacterium accolens SU004, Corynebacterium tuberculostearicum SU005 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU004, Corynebacterium accolens SU009 and Corynebacterium propinquum SU010, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU009 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU004, Corynebacterium tuberculostearicum SU005 and Corynebacterium accolens SU006, or any derivative, variant or mutant thereof capable of reducing cathepsin expression. In another preferred embodiment, the combination comprises at least three strains, wherein the at least three strains are Corynebacterium accolens SU004, Corynebacterium accolens SU006 and Corynebacterium propinquum SU010, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU006 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU004, Corynebacterium tuberculostearicum SU005 and Corynebacterium propinquum SU007, or any derivative, variant or mutant thereof capable of reducing cathepsin expression. In another preferred embodiment, the combination comprises at least three strains, wherein the at least three strains are Corynebacterium accolens SU004, Corynebacterium tuberculostearicum SU005 and Corynebacterium accolens SU008, or any derivative, variant or mutant thereof capable of reducing cathepsin expression. In another preferred embodiment, the combination comprises at least three strains, wherein the at least three strains are Corynebacterium accolens SU004, Corynebacterium propinquum SU007 and Corynebacterium accolens SU008, or any derivative, variant or mutant thereof capable of reducing cathepsin expression. In another preferred embodiment, the combination comprises at least three strains, wherein the at least three strains are Corynebacterium tuberculostearicum SU005, Corynebacterium propinquum SU007 and Corynebacterium accolens SU008, or any derivative, variant or mutant thereof capable of reducing cathepsin expression. In another preferred embodiment, the combination comprises at least three strains, wherein the at least three strains are Corynebacterium accolens SU004, Corynebacterium accolens SU006 and Corynebacterium propinquum SU007, or any derivative, variant or mutant thereof capable of reducing cathepsin expression. In another preferred embodiment, the combination comprises at least three strains, wherein the at least three strains are Corynebacterium accolens SU004, Corynebacterium accolens SU006 and Corynebacterium accolens SU008, or any derivative, variant or mutant thereof capable of reducing cathepsin expression. In another preferred embodiment, the combination comprises at least three strains, wherein the at least three strains are Corynebacterium accolens SU004, Corynebacterium accolens SU006 and Corynebacterium accolens SU009, or any derivative, variant or mutant thereof capable of reducing cathepsin expression. In another preferred embodiment, the combination comprises at least three strains, wherein the at least three strains are Corynebacterium accolens SU004, Corynebacterium propinquum SU007 and Corynebacterium accolens SU009, or any derivative, variant or mutant thereof capable of reducing cathepsin expression. In another preferred embodiment, the combination comprises at least three strains, wherein the at least three strains are Corynebacterium accolens SU004, Corynebacterium propinquum SU007 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU004, Corynebacterium accolens SU008 and Corynebacterium accolens SU009, or any derivative, variant or mutant thereof capable of reducing cathepsin expression. In another preferred embodiment, the combination comprises at least three strains, wherein the at least three strains are Corynebacterium accolens SU004, Corynebacterium accolens SU008 and Corynebacterium propinquum SU010, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU006 and Corynebacterium propinquum SU007, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU006 and Corynebacterium accolens SU008, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU006 and Corynebacterium accolens SU009, or any derivative, variant or mutant thereof capable of reducing cathepsin expression. In another preferred embodiment, the combination comprises at least three strains, wherein the at least three strains are Corynebacterium tuberculostearicum SU005, Corynebacterium propinquum SU007 and Corynebacterium accolens SU009, or any derivative, variant or mutant thereof capable of reducing cathepsin expression. In another preferred embodiment, the combination comprises at least three strains, wherein the at least three strains are Corynebacterium tuberculostearicum SU005, Corynebacterium propinquum SU007 and Corynebacterium propinquum SU010, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU008 and Corynebacterium accolens SU009, or any derivative, variant or mutant thereof capable of reducing cathepsin expression. In another preferred embodiment, the combination comprises at least three strains, wherein the at least three strains are Corynebacterium tuberculostearicum SU005, Corynebacterium accolens SU008 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU006, Corynebacterium propinquum SU007 and Corynebacterium accolens SU008, or any derivative, variant or mutant thereof capable of reducing cathepsin expression. In another preferred embodiment, the combination comprises at least three strains, wherein the at least three strains are Corynebacterium accolens SU006, Corynebacterium propinquum SU007 and Corynebacterium accolens SU009, or any derivative, variant or mutant thereof capable of reducing cathepsin expression. In another preferred embodiment, the combination comprises at least three strains, wherein the at least three strains are Corynebacterium accolens SU006, Corynebacterium propinquum SU007 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU006, Corynebacterium accolens SU008 and Corynebacterium accolens SU009, or any derivative, variant or mutant thereof capable of reducing cathepsin expression. In another preferred embodiment, the combination comprises at least three strains, wherein the at least three strains are Corynebacterium accolens SU006, Corynebacterium accolens SU008 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU006, Corynebacterium accolens SU009 and Corynebacterium propinquum SU010, or any derivative, variant or mutant thereof capable of reducing cathepsin expression. In another preferred embodiment, the combination comprises at least three strains, wherein the at least three strains are Corynebacterium propinquum SU007, Corynebacterium accolens SU008 and Corynebacterium accolens SU009, or any derivative, variant or mutant thereof capable of reducing cathepsin expression. In another preferred embodiment, the combination comprises at least three strains, wherein the at least three strains are Corynebacterium propinquum SU007, Corynebacterium accolens SU008 and Corynebacterium propinquum SU010, or any derivative, variant or mutant thereof capable of reducing cathepsin expression. In another preferred embodiment, the combination comprises at least three strains, wherein the at least three strains are Corynebacterium propinquum SU007, Corynebacterium accolens SU009 and Corynebacterium propinquum SU010, or any derivative, variant or mutant thereof capable of reducing cathepsin expression. In another preferred embodiment, the combination comprises at least three strains, wherein the at least three strains are Corynebacterium accolens SU008, Corynebacterium accolens SU009 and Corynebacterium propinquum SU010, or any derivative, variant or mutant thereof capable of reducing cathepsin expression.

In a preferred embodiment, the combination comprises four strains, wherein the four strains are Corynebacterium accolens SU004, Corynebacterium tuberculostearicum SU005, Corynebacterium accolens SU006 and Corynebacterium propinquum SU007, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU006 and Corynebacterium accolens SU008, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU006 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU006 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium propinquum SU007 and Corynebacterium accolens SU008, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium propinquum SU007 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium propinquum SU007 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium tuberculostearicum SU005, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium propinquum SU007 and Corynebacterium accolens SU008, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium propinquum SU007 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium propinquum SU007 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium accolens SU006, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium propinquum SU007, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium accolens SU004, Corynebacterium propinquum SU007, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium propinquum SU007, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium accolens SU004, Corynebacterium accolens SU008, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium propinquum SU007 and Corynebacterium accolens SU008, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium propinquum SU007 and Corynebacterium accolens SU009, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium propinquum SU007 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium accolens SU006, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium propinquum SU007, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium tuberculostearicum SU005, Corynebacterium propinquum SU007, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium propinquum SU007, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium tuberculostearicum SU005, Corynebacterium accolens SU008, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium accolens SU006, Corynebacterium propinquum SU007, Corynebacterium accolens SU008 and Corynebacterium accolens SU009, or any derivative, variant or mutant thereof capable of reducing cathepsin expression. In another preferred embodiment, the combination comprises four strains, wherein the four strains are Corynebacterium accolens SU006, Corynebacterium propinquum SU007, Corynebacterium accolens SU008 and Corynebacterium propinquum SU010, or any derivative, variant or mutant thereof capable of reducing cathepsin expression. In another preferred embodiment, the combination comprises four strains, wherein the four strains are Corynebacterium accolens SU006, Corynebacterium propinquum SU007, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium accolens SU006, Corynebacterium accolens SU008, Corynebacterium accolens SU009 and Corynebacterium propinquum SU010, or any derivative, variant or mutant thereof capable of reducing cathepsin expression. In another preferred embodiment, the combination comprises four strains, wherein the four strains are Corynebacterium propinquum SU007, Corynebacterium accolens SU008, Corynebacterium accolens SU009 and Corynebacterium propinquum SU010, or any derivative, variant or mutant thereof capable of reducing cathepsin expression.

In a more preferred embodiment, the Corynebacterium culture comprises four Corynebacterium strains selected from:
(i) Corynebacterium accolens SU004, Corynebacterium tuberculostearicum SU005, Corynebacterium accolens SU009 and Corynebacterium propinquum SU010, or any derivative, variant or mutant thereof capable of reducing cathepsin expression;
(ii) Corynebacterium accolens SU004, Corynebacterium tuberculostearicum SU005, Corynebacterium accolens SU006, and Corynebacterium propinquum SU010, or any derivative, variant or mutant thereof capable of reducing cathepsin expression; and
(iii) Corynebacterium accolens SU004, Corynebacterium tuberculostearicum SU005, Corynebacterium propinquum SU007 and Corynebacterium accolens SU008, or any derivative, variant or mutant thereof capable of reducing cathepsin expression.

In a preferred embodiment, the combination comprises four strains, wherein the fours strains are Corynebacterium accolens SU004, Corynebacterium tuberculostearicum SU005, Corynebacterium accolens SU009 and Corynebacterium propinquum SU010, or any derivative, variant or mutant thereof capable of reducing cathepsin expression. (This combination is called combination K1 in the Examples.) In another preferred embodiment, the combination comprises four strains, wherein the fours strains are Corynebacterium accolens SU004, Corynebacterium tuberculostearicum SU005, Corynebacterium accolens SU006 and Corynebacterium propinquum SU010, or any derivative, variant or mutant thereof capable of reducing cathepsin expression. (This combination is called combination K2 in the Examples.) In another preferred embodiment, the combination comprises four strains, wherein the fours strains are Corynebacterium accolens SU004, Corynebacterium tuberculostearicum SU005, Corynebacterium propinquum SU007 and Corynebacterium accolens SU008, or any derivative, variant or mutant thereof capable of reducing cathepsin expression. (This combination is called combination K3 in the Examples.)

Preferably, the Corynebacterium culture comprising at least two Corynebacterium strains consists essentially of Corynebacterium, preferably essentially of Corynebacterium accolens and/or Corynebacterium propinquum and/or Corynebacterium tuberculostearicum.

In particular, the Corynebacterium culture comprising at least two Corynebacterium strains comprises at least two viable Corynebacterium strains in a medium. Preferably, the Corynebacterium culture comprising at least two Corynebacterium strains comprises at least three viable Corynebacterium strains in a medium. More preferably, the Corynebacterium culture comprising at least two Corynebacterium strains comprises four viable Corynebacterium strains in a medium.

In a preferred embodiment of the invention, the Corynebacterium culture comprising at least two Corynebacterium strains comprises at least two viable Corynebacterium strains selected from the group of strains of Corynebacterium accolens, Corynebacterium propinquum, and Corynebacterium tuberculostearicum. In a preferred embodiment of the invention, the Corynebacterium culture comprising at least two Corynebacterium strains comprises at least three viable Corynebacterium strains selected from the group of strains of Corynebacterium accolens, Corynebacterium propinquum, and Corynebacterium tuberculostearicum. In a preferred embodiment of the invention, the Corynebacterium culture comprising at least two Corynebacterium strains comprises four viable Corynebacterium strains selected from the group of strains of Corynebacterium accolens, Corynebacterium propinquum, and Corynebacterium tuberculostearicum.

In any one of the aspects of the invention, the Corynebacterium culture does not comprise Corynebacterium accolens SU001 and/or Corynebacterium propinquum SU002 and/or Corynebacterium propinquum SU003. In an embodiment, the Corynebacterium culture does not comprise Corynebacterium accolens SU001. In another embodiment, the Corynebacterium culture does not comprise Corynebacterium propinquum SU002. In another embodiment, the Corynebacterium culture does not comprise Corynebacterium propinquum SU003.

In any one of the aspects of the invention, preferably the Corynebacterium culture does not comprise Corynebacterium pseudodiphtheriticum.

Preferably, in any aspects of the invention, the Corynebacterium strain or Corynebacterium culture that is capable of reducing the expression of a cathepsin is a Corynebacterium strain or Corynebacterium culture, respectively that reduces the relative expression of cathepsin mRNA after 24-hour incubation with Caco-2 cells. In an embodiment, the bacterial co-culture examination described in the Examples is used to determine whether a Corynebacterium strain or Corynebacterium culture reduces the relative expression of cathepsin mRNA.

The invention also relates to a pharmaceutical composition comprising a Corynebacterium culture capable of reducing the expression of a cathepsin in a subject, and a pharmaceutically acceptable excipient or pharmaceutically acceptable carrier.

Preferably, the formulation or composition is a pharmaceutical composition comprising a pharmaceutically acceptable excipient. Preferably, the formulation or composition is a probiotic formulation or composition. Preferably, the formulation or the composition is (part of) a medical device. Preferably, the formulation or the composition is delivered by a medical device. Preferably, the formulation or composition is for inhalative administration, more preferably in the form of inhalable powders, aerosol mixtures, oral inhalation solutions or suspensions, nasal drops, nasal sprays, nasopharyngeal sprays, or nasal mists. Preferably, the formulation or composition is a nasal spray.

The invention also relates to a formulation comprising the Corynebacterium culture for use according to the invention. The formulation of the invention comprises a pharmaceutically acceptable excipient, e.g. a carrier. Preferably, the formulation comprises one or more ingredients which support Corynebacterium viability or propagation, if desired, growth, and such ingredient optionally considered as excipient. Any excipient is preferably physiologically compatible with the upper respiratory tract epithelium.

Preferably, the formulation is for use in the upper respiratory tract of a subject.

In an embodiment, the formulation is for nasal administration, more preferably nasal drops or nasal spray.

In an embodiment, the formulation is for inhalative administration, more preferably in the form of inhalable powders, aerosol mixtures, oral inhalation solutions or suspensions, nasal sprays, nasal mists.

In an embodiment, the formulation is in the form of an intranasal formulation for topical administration, preferably in the form of viscous liquid, a liquid suspension, a paste, a gel or an ointment.

Preferably, the formulation is a formulation delivered to the upper respiratory tract. Preferably, the formulation is delivered by a device capable of delivering a formulation to the upper respiratory tract.

Preferably, the formulation is in the form of a nasal preparation, for example nasal cream, nasal gel, nasal ointment, nasal drops (including solution, suspension, emulsion, lyophilizate for suspension, and powder for solution), nasal powder, nasal spray (including solution, suspension, emulsion, lyophilizate for suspension, and powder for solution), nasal wash, nasal stick or nasopharyngeal spray. Optionally, the formulation is in the form of a cutaneous and nasal ointment, endosinusial solution, endosinusinal wash (including suspension), powder for endosinusial solution, powder for solution for nasal spray, or an oromucosal solution, nasal/oromucosal solution, or nasal/oromucosal spray.

Preferably, the formulation is in the form of an oropharyngeal preparation, for example an oropharyngeal spray, oropharyngeal spray solution, oropharyngeal suspension, oropharyngeal emulsion, oropharyngeal powder, oropharyngeal gel or oropharyngeal wash.

Optionally, the formulation is in the form of an oculonasal preparation, for example effervescent tablet for oculonasal suspension, lyophilizate for oculonasal suspension (including use in drinking water), or oculonasal suspension.

Preferably, the formulation is in the form of an oromucosal preparation, for example buccal film, buccal tablet, compressed lozenge, concentrate for gargle, concentrate for oromucosal solution, cutaneous solution, cutaneous/oromucosal solution, cutaneous/oromucosal spray, cutaneous/oromucosal/oral solution, effervescent buccal tablet, gargle (including powder for solution, tablet for solution), gargle/mouthwash, gargle/nasal wash, gingival gel, gingival paste, gingival solution, laryngopharyngeal solution, laryngopharyngeal spray, lozenge, medicated chewing-gum, mouthwash (including powder for solution, tablet for solution), muco-adhesive buccal prolonged-release tablet, muco-adhesive buccal tablet, nasal spray and oromucosal solution, nasal/oromucosal solution, nasal/oromucosal spray, oromucosal capsule, oromucosal cream, oromucosal drops, oromucosal film, oromucosal gel, oromucosal ointment, oromucosal paste, oromucosal patch, oromucosal pouch, oromucosal solution, oromucosal spray (including emulsion, solution and suspension), oromucosal suspension, oromucosal/laryngopharyngeal solution, oromucosal/laryngopharyngeal spray, pastille, pillules, powder for gingival gel, powder for mouth wash, sublingual film, sublingual lyophilizate, sublingual powder, sublingual spray (including emulsion, solution, and suspension), or sublingual tablet.

Preferably, the formulation is in the form of a pulmonary preparation, for example aerosol, concentrate for nebuliser solution, endotracheopulmonary instillation (including solution, suspension, powder for solution, and powder for suspension), inhalation gas, inhalation impregnated pad, inhalation powder (including hard capsule, pre-dispensed, and tablet), inhalation solution, inhalation vapour (including capsule, effervescent tablet, emulsion, impregnated pad, impregnated plug, liquid, ointment, powder, solution, and tablet), liquefied gas for dental use, medicinal gas (including compressed, cryogenic, and liquefied), nebuliser emulsion, nebuliser solution, nebuliser suspension, oral solution/concentrate for nebuliser solution, powder for nebuliser solution, powder for nebuliser solution/solution for injection/infusion, powder for nebuliser suspension, pressurized inhalation (including emulsion, solution and suspension), metered-dose inhalation.

The invention also relates to a lyophilized formulation comprising a Corynebacterium culture capable of reducing the expression of a cathepsin in a subject, and a cryoprotectant.

The invention also relates to a formulation comprising any of the Corynebacterium cultures (including combinations) defined above, wherein the formulation is a lyophilized formulation. The lyophilized formulation comprises any of the Corynebacterium cultures (including combinations) defined above, and a cryoprotectant.

In an embodiment, in said lyophilized formulation, the cryoprotectant comprises a hydrocolloid polymer and/or a carbohydrate or carbohydrate derivative. Preferably, in said lyophilized formulation, the cryoprotectant comprises a hydrocolloid polymer and a carbohydrate or carbohydrate derivative.

Preferably, in said lyophilized formulation, the carbohydrate or carbohydrate derivative is selected from trehalose, sucrose, glucose, lactose, mannitol, sorbitol, inulin, maltodextrin and isomaltulose, preferably from maltodextrin and isomaltulose. Preferably, in said lyophilized formulation, the carbohydrate is isomaltulose.

Preferably, in said lyophilized formulation, the carbohydrate or carbohydrate derivative is maltodextrin in a concentration of 0.1 g/100 mL to 3 g/100 mL, preferably in a concentration of 0.5 g/100 mL to 2.5 g/100 mL, or isomaltulose in a concentration of 0.1 g/100 mL to 3 g/100 mL, preferably in a concentration of 0.5 g/100 mL to 2.5 g/100 mL. Preferably, in said lyophilized formulation, the carbohydrate is isomaltulose in a concentration of 0.5 g/100 mL to 2.5 g/100 mL, more preferably in a concentration of 0.5 g/100 mL.

Preferably, in said lyophilized formulation, the hydrocolloid polymer is selected from hydroxypropyl methylcellulose, hydroxy-ethyl-cellulose, carboxymethylcellulose, guar gum, carrageenan and xanthan, preferably from kappa carrageenan and xanthan. Optionally, the hydrocolloid polymer is a pH-modifying hydrocolloid polymer (e.g., alginic acid/alginate, poly-galactomannuronic acid), in which case a buffer is added to the formulation.

Preferably, in said lyophilized formulation, the hydrocolloid polymer is kappa carrageenan in a concentration of 0.05 g/100 mL to 1 g/100 mL, preferably in a concentration of 0.1 g/100 mL to 0.5 g/100 mL, or xanthan in a concentration of 0.01 g/100 mL to 0.5 g/100 mL, preferably in a concentration of 0.05 g/100 mL to 0.25 g/100 mL. Preferably, in said lyophilized formulation, the hydrocolloid polymer is xanthan in a concentration of 0.05 g/100 mL to 0.25 g/100 mL, more preferably in a concentration of 0.05 g/100 mL. Preferably, in said lyophilized formulation, the hydrocolloid polymer is kappa carrageenan in a concentration of 0.1 g/100 mL to 0.5 g/100 mL, more preferably in a concentration of 0.1 g/100 mL.

In a preferred embodiment, the lyophilized formulation comprises a Corynebacterium culture comprising four Corynebacterium strains selected from:
(i) Corynebacterium accolens SU004, Corynebacterium tuberculostearicum SU005, Corynebacterium accolens SU009 and Corynebacterium propinquum SU010, or any derivative, variant or mutant thereof capable of reducing cathepsin expression;
(ii) Corynebacterium accolens SU004, Corynebacterium tuberculostearicum SU005, Corynebacterium accolens SU006, and Corynebacterium propinquum SU010, or any derivative, variant or mutant thereof capable of reducing cathepsin expression; and
(iii) Corynebacterium accolens SU004, Corynebacterium tuberculostearicum SU005, Corynebacterium propinquum SU007 and Corynebacterium accolens SU008, or any derivative, variant or mutant thereof capable of reducing cathepsin expression; and
comprises a carbohydrate or carbohydrate derivative, and a hydrocolloid polymer;
preferably wherein the carbohydrate or carbohydrate derivative is maltodextrin or isomaltulose, and wherein the hydrocolloid polymer is kappa carrageenan or xanthan.

Preferably, the lyophilized formulation comprises any of the Corynebacterium cultures (including combinations) defined above, wherein the Corynebacterium culture comprises 10³ CFU/mL to 10¹¹ CFU/mL of Corynebacterium strain(s). Preferably, the Corynebacterium culture comprises 10⁴ CFU/mL to 10¹¹ CFU/mL, preferably 10⁵ CFU/mL to 10¹¹ CFU/mL, preferably 10⁶ CFU/mL to 10¹¹ CFU/mL, preferably 10⁷ CFU/mL to 10¹¹ CFU/mL, preferably 10⁸ CFU/mL to 10¹¹ CFU/mL, preferably 10⁸ CFU/mL to 10¹⁰ CFU/mL, preferably 10⁹ CFU/mL to 10¹¹ CFU/mL, preferably 10⁹ CFU/mL to 10¹⁰ CFU/mL of Corynebacterium strain(s), more preferably 10⁸ CFU/mL or 10⁹ CFU/mL of Corynebacterium strain(s). Preferably, the Corynebacterium culture comprises at least two, at least three, or four Corynebacterium strains, wherein the culture comprises 10⁴ CFU/mL to 10¹¹ CFU/mL of each strain. More preferably, the Corynebacterium culture comprises 10⁷ CFU/mL to 10¹¹ CFU/mL, preferably 10⁸ CFU/mL to 10¹¹ CFU/mL, preferably 10⁸ CFU/mL to 10¹⁰ CFU/mL, preferably 10⁹ CFU/mL to 10¹¹ CFU/mL, preferably 10⁹ CFU/mL to 10¹⁰ CFU/mL of each Corynebacterium strain, preferably 10⁸ CFU/mL or 10⁹ CFU/mL of each Corynebacterium strain.

In another aspect, the invention relates to a kit comprising any of the lyophilized formulation described above and a suitable reconstitution agent (e.g., Ringer's solution). The reconstitution agent is used to reconstitute the lyophilized formulation into a formulation for use in the upper respiratory tract of a subject. Preferably, the reconstitution agent is used to reconstitute the lyophilized formulation into a nasal spray. The reconstitution agent is preferably Ringer's solution.

The invention relates to a Corynebacterium culture capable of reducing the expression of a cathepsin in a subject for use in medicine.

The invention relates to Corynebacterium culture or lyophilized formulation for use in the prevention or treatment of an infection by a respiratory virus in a subject, said Corynebacterium culture being capable of reducing the expression of a cathepsin in the subject. In another embodiment, the invention relates to Corynebacterium culture or lyophilized formulation for use in the prevention of an infection by an enveloped respiratory virus in a subject, wherein said virus uses cathepsin entry route for entry into the cell of the subject and wherein the Corynebacterium culture is capable of reducing the expression of a cathepsin in the subject. Preferably, the invention relates to Corynebacterium culture or lyophilized formulation for use in the prevention or treatment of an infection by an enveloped respiratory virus in a subject, said Corynebacterium culture being capable of reducing the expression of a cathepsin in the subject. The enveloped respiratory virus is a DNA or RNA virus, preferably an RNA virus. Preferably, the invention relates to Corynebacterium culture or lyophilized formulation for use in the prevention or treatment of an infection by a coronavirus in a subject, said Corynebacterium culture being capable of reducing the expression of a cathepsin in the subject, wherein the coronavirus is preferably SARS-CoV-2, more preferably a SARS-CoV-2 variant that primarily uses the cathepsin B/L entry route, most preferably SARS-CoV-2 omicron variant.

The invention also relates to a Corynebacterium culture or lyophilized formulation for use in the prevention or treatment of an infection by a respiratory virus, preferably an enveloped respiratory virus, more preferably a coronavirus, most preferably a SARS-CoV-2 virus in a subject, said Corynebacterium culture being capable of reducing the expression of a cathepsin in the subject, and optionally also being capable of
reducing expression of ACE2 in cells of the subject, and/or
reducing expression of TMPRSS2 in cells of the subject.

The invention also relates to a Corynebacterium culture or lyophilized formulation for use in the prevention or treatment of an infection by a respiratory virus, preferably an enveloped respiratory virus, more preferably a coronavirus, most preferably a SARS-CoV-2 virus in a subject, said Corynebacterium culture being capable of reducing the expression of a cathepsin in the subject, and optionally also being capable of
reducing expression of ACE2 in cells of the subject, and/or
reducing expression of TMPRSS2 in cells of the subject, and/or
inhibiting the binding of ACE2 receptor and spike (S) protein of the virus.

In an alternative embodiment the invention relates to a Corynebacterium culture (including combinations) or lyophilized formulation for use in the upper respiratory tract of a homeothermic subject, against a pathogenic respiratory RNA virus infection of said subject, said Corynebacterium culture being capable of reducing the expression of a cathepsin in cells of the subject. In another embodiment, the Corynebacterium culture (including combinations) or lyophilized formulation is for use in the upper respiratory tract of a homeothermic subject, against an enveloped respiratory virus, preferably a coronavirus, more preferably a SARS-CoV-2 virus.

In a preferred embodiment, the Corynebacterium culture or lyophilized formulation is for use in the upper respiratory tract of the subject.

In an embodiment, the Corynebacterium culture further comprises of a nucleotide sequence encoding a lipase enzyme. Preferably, the Corynebacterium culture is further capable of producing a lipase enzyme.

The subject is a vertebrate subject selected from fishes, amphibians, reptiles, birds, and mammals, preferably from reptiles, birds and mammals. In an embodiment the subject is a homeothermic subject selected from birds and mammals. Preferably, the subject is a mammalian subject, preferably human subject.

In particular the subject is a mammalian or avian, preferably mammalian. In particular, the subject is a mammalian subject, preferably a farm animal, a domestic animal or a human, in particular a human subject.

Preferably, the pathogenic respiratory RNA virus or the enveloped respiratory virus is a coronavirus. Preferably the virus is a SARS coronavirus. Preferably the virus is selected from SARS-CoV-1, SARS-CoV-2 and MERS. Preferably, the coronavirus is SARS-CoV-2. More preferably, the coronavirus is a SARS-CoV-2 variant that uses cathepsin entry route. More preferably, the coronavirus is a SARS-CoV-2 variant that primarily uses the cathepsin B/L entry route. Most preferably, the coronavirus is SARS-CoV-2 omicron variant.

Preferably, the pathogenic respiratory RNA virus or the enveloped respiratory virus is a virus that uses cathepsin entry route for entry into the cell of the subject.

In particular, the Corynebacterium culture is any Corynebacterium culture defined above, including combinations.

In a preferred embodiment, the Corynebacterium culture or lyophilized formulation is for use in the prevention of a respiratory tract infection, respiratory tract colonization or respiratory tract disease. In a preferred embodiment, the Corynebacterium culture or lyophilized formulation is for use in the prevention of a disease caused by an infectious agent that settles in the respiratory tract, preferably in the upper respiratory tract.

In a preferred embodiment, the Corynebacterium culture or lyophilized formulation is for use in the prevention of a disease caused by a respiratory RNA virus infection. The respiratory virus causes respiratory tract infections.

In a preferred embodiment, the Corynebacterium culture or lyophilized formulation for use according to the invention is for use against an enveloped coronavirus having a spike protein, and wherein said Corynebacterium culture is capable of reducing expression of a cathepsin. In a preferred embodiment, the Corynebacterium culture or lyophilized formulation for use according to the invention is for use against an enveloped coronavirus having a spike protein, and wherein said Corynebacterium culture is capable of reducing expression of a cathepsin and is also capable of
reducing expression of a receptor protein, preferably ACE2 in host cells, preferably epithelial cells, and/or
reducing expression of a serine protease activating the spike protein, preferably TMPRSS2 in host cells, preferably epithelial cells, and optionally,
reducing/inhibiting the interaction between the spike protein and the receptor protein, preferably ACE2 in host cells.

In a preferred embodiment, the RNA virus is an enveloped coronavirus having a spike protein (preferably a coronavirus selected from SARS-CoV-1, SARS-CoV-2 and MERS), and reducing expression of a cathepsin comprises reducing expression of mRNA of the cathepsin and/or reducing expression of the cathepsin at protein level. In a preferred embodiment, the RNA virus is an enveloped coronavirus having a spike protein (preferably a coronavirus selected from SARS-CoV-1, SARS-CoV-2 and MERS), and reducing expression of a cathepsin comprises reducing expression of mRNA of the cathepsin and/or reducing expression of the cathepsin at protein level, and optionally,
reducing expression of a receptor protein (preferably ACE2) in host cells comprises reducing expression of mRNA of the receptor protein and/or reducing expression of a receptor protein at protein level, and/or
reducing expression of a serine protease activating the spike protein (preferably TMPRSS2) in host cells comprises reducing expression of mRNA of the receptor protein and/or reducing expression of a receptor protein at protein level.

In a preferred embodiment of the invention, the Corynebacterium culture (including combinations) or lyophilized formulation is for use in the treatment or prevention of a respiratory disease of the upper respiratory tract. In a preferred embodiment, the Corynebacterium culture (including combinations) or lyophilized formulation is for use in the treatment or prevention of a respiratory tract infection, respiratory tract colonization or respiratory tract disease. Preferably, the respiratory tract infection, the respiratory tract colonization or the respiratory tract disease is of the upper respiratory tract. In a preferred embodiment, the Corynebacterium culture (including combinations) or lyophilized formulation is for use in the prevention of a disease caused by an infectious agent that settles in the respiratory tract, preferably in the upper respiratory tract.

Preferably said Corynebacterium culture (including combinations) or said lyophilized formulation is administered to the upper respiratory tract of said subject,
preferably to the nasal cavity and the pharynx, preferably to the nasopharynx, oropharynx, oro/mesopharynx, and/or laryngopharynx, in particular to the nasopharynx,
preferably in the form of drops, spray, inhalable formulation or ointment.

Preferably, the Corynebacterium culture (including combinations) or lyophilized formulation is for use in the treatment including prevention, or preferably prevention of a respiratory disease caused by a coronavirus, preferably a SARS virus in a subject. Preferably the coronavirus is selected from SARS-CoV-1, SARS-CoV-2 and MERS. More preferably, the coronavirus is a SARS-CoV-2 variant that primarily uses the cathepsin B/L entry route, preferably SARS-CoV-2 omicron variant. Preferably the respiratory disease is COVID-19 disease. Preferably the subject is a mammalian subject, preferably a human subject.

Preferably, the Corynebacterium culture or lyophilized formulation is for use in the prevention of the symptoms of a disease caused by an infectious agent that settles in the respiratory tract, preferably in the upper respiratory tract. Preferably, the Corynebacterium culture or lyophilized formulation is for use in the prevention of the symptoms of a disease caused by a coronavirus. These symptoms include fever or chills, cough, shortness of breath or difficulty breathing, fatigue, muscle or body aches, headache, loss of taste or smell, sore throat, congestion or runny nose, nausea, vomiting, and diarrhea.

In a preferred embodiment, the Corynebacterium culture or combination, or lyophilized formulation thereof is administered to a subject for about 5 to 7 days, once or twice daily. In a preferred embodiment, the Corynebacterium culture or combination, or lyophilized formulation thereof is administered to a subject twice a day, preferably in the morning and in the evening. In case when the administration is into the throat (e.g. by a gargle or mouthwash formulation), the Corynebacterium culture or combination, or lyophilized formulation thereof is administered to a subject after a meal.

The invention also relates to a method for the prevention of an infection by an enveloped respiratory virus, preferably a coronavirus, more preferably SARS-CoV-2, more preferably SARS-CoV-2 omicron variant, said method comprising
administering a Corynebacterium culture as defined above, or a formulation as defined above, or a composition as defined above
to a subject in need thereof.

Preferably, the method comprises administering a Corynebacterium culture comprising at least one or at least two, preferably at least three, more preferably four Corynebacterium strains selected from
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16, Hungary) on Jan. 17, 2022, or a derivative, variant or mutant thereof, wherein the derivative, variant or mutant thereof is capable of reducing cathepsin expression;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression; or
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression.

Preferably, the method comprises administering a lyophilized formulation described above.

### ABBREVIATIONS

ACE2 Angiotensin I Converting Enzyme 2
CFU Colony Forming Unit
COVID-19 coronavirus disease 2019
EMEM Eagle's minimal essential medium
GAPDH Glyceraldehyde-3-phosphate dehydrogenase
MALDI-TOF Matrix-assisted laser desorption-ionization time of flight
MERS Middle East respiratory syndrome
NCAIM National Collection of Agricultural and Industrial Microorganisms
PBS Phosphate-buffered saline
PCR Polymerase chain reaction
rRNA ribosomal RNA
SARS-CoV-2 Severe acute respiratory syndrome coronavirus 2
TMPRSS2 transmembrane protease/serine subfamily member 2

### DEFINITIONS

As used herein bacteria are “viable” if, under conditions which comprise an appropriate medium and nutrients and appropriate temperature, they are capable of propagation (“growth”, if the number of the viable bacteria increases).

A “culture”, as used herein, refers to the cultivation of bacteria in an artificial environment, i.e. in vitro. Cultivation and thus culturing may include maintaining the bacteria in a viable form and/or propagation of the bacteria. Thus “culture” includes bacteria in a medium in which the bacteria are maintained in a viable form, including dried (lyophilized) forms, cultures on solid media and liquid forms as well. The term culture also encompasses the meaning of the term “consortium”. A “culture”, as used herein, may comprise only one strain, or may contain more than one strains. Accordingly, as used herein, the term “Corynebacterium culture” is a culture that comprises viable Corynebacteria. The culture can comprise only one Corynebacterium strain, or it can contain at least two (e.g., at least three, preferably four) different Corynebacterium strains, unless indicated otherwise.

Preferably, a Corynebacterium strain or Corynebacterium culture that is “capable of reducing the expression of a protein” is a Corynebacterium strain or Corynebacterium culture, respectively that reduces said protein expression in a mammalian cell in an in vitro co-culture of the mammalian cells and the Corynebacterium bacteria. Preferably, the expression is determined by measuring mRNA levels of said protein and the expression of the protein is “reduced” when the mRNA levels of the protein in the mammalian cells of the co-culture are lower compared to the mRNA levels of the protein measured in control mammalian cells (without bacteria). Preferably, the mammalian cell is a human cell, optionally a human colonic epithelial cell line Caco-2. In an embodiment, the Corynebacterium strain or Corynebacterium culture that is capable of reducing the expression of a cathepsin is a Corynebacterium strain or Corynebacterium culture, respectively that reduces the relative expression of cathepsin mRNA after 24-hour incubation with Caco-2 cells.

The term “formulation” relates to a composition of matter comprising at least one biologically, preferably medically active ingredient, the “active agent”, and at least one other substance e.g. a medium and/or excipient or both suitable for administration into a subject, e.g. a mammal or bird. Preferred formulations are formulations for use in the upper respiratory tract of said subject, preferably a homeothermic subject. The term “formulation” and the term “composition” may be used interchangeably in the context of the invention.

The term “administration” as used herein shall include routes of introducing or applying formulation of the invention to a subject in need thereof to perform their intended function. In particular, administration as used herein relates to administration into the upper respiratory tract of a subject, e.g. a mammal or a bird.

The term “subject” as used herein shall refer to a homeothermic (mammalian or avian, preferably mammalian) subject, particularly a human being. In particular, the medical use of the invention or the respective method of treatment applies to a subject in need of prophylaxis or treatment of a disorder or disease caused by an infectious agent that settles in the respiratory tract, preferably in the upper respiratory tract.

The term “patient” includes a subject under medical care, e.g. one that receives either prophylactic or therapeutic treatment.

“Prophylactic treatment” or “prophylaxis” or “prevention”, as used herein includes measure or measures on or in respect of a patient to prevent infection of the patient including reduction of infection rate or severity of infection (number of infected cells). The patient may be a non-infected patient or a patient who has been infected but further infection or reinfection is prevented by said measure or measures. Preferably prophylaxis or prevention includes at least one or more or regular administration to a target site, i.e. the upper respiratory tract, in particular the epithelium thereof.

“Treating”, “to treat”, “treatment” as used herein refer to improving, alleviating, reducing, mitigating a symptom of a disease or condition, e.g. a symptom of an infection. “Treating”, “to treat”, “treatment” also include reducing the number of viruses which may enter the cells of the patient.

The “upper respiratory tract” refers to the parts of the respiratory system lying above the sternal angle (outside of the thorax) above the vocal folds, or above the cricoid cartilage. Preferably, the larynx is sometimes included in the upper respiratory tract. The upper respiratory tract comprises the nasal cavity and paranasal sinuses, the pharynx (including the nasopharynx/epipharynx, oropharynx/mesopharynx and laryngopharynx/hypopharynx) and preferably includes the larynx. In preferred embodiments, “upper respiratory tract” refers to the nasal cavity and the nasopharynx.

The expression “infection” is intended to mean an undesired propagation of virus in the cells of a subject.

The term “comprise(s)” or “comprising” or “including” are to be construed herein as having a non-exhaustive meaning and to allow the addition or involvement of further features or method steps or components to anything which comprises the listed features or method steps or components. Such terms can be limited to “consisting essentially of” or “comprising substantially” which is to be understood as consisting of mandatory features or method steps or components listed in a list, e.g. in a claim, whereas allowing to contain additionally other features or method steps or components which do not materially affect the essential characteristics of the use, method, composition or other subject matter.

As used in this specification and the appended claims, the singular forms “a”, “an” and “the” include plural references, and should be construed as including the meaning “one or more”, unless the content clearly dictates otherwise. In general, it is also to be understood that the terminology used herein is for the purpose of describing particular embodiments only, and is not intended to be limiting.

The “formulation for use in the upper respiratory tract” includes any formulations delivered to the upper respiratory tract of a subject, including any nasal, nasopharyngeal, oculonasal, oromucosal and pulmonary formulations listed above.

The term “Corynebacterium accolens SU001” refers to the Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jun. 22, 2021.

The term “Corynebacterium propinquum SU002” refers to the Corynebacterium propinquum SU002 strain having the accession number NCAIM P (B) 001496 deposited at NCAIM on Jun. 22, 2021.

The term “Corynebacterium propinquum SU003” refers to the Corynebacterium propinquum SU003 strain having the accession number NCAIM P (B) 001497 deposited at NCAIM on Jun. 22, 2021.

The term “Corynebacterium accolens SU004” refers to the Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16., Hungary) on Jan. 17, 2022.

The term “Corynebacterium tuberculostearicum SU005” refers to the Corynebacterium tuberculostearicum SU005 strain having the accession number NCAIM P (B) 001501 deposited at NCAIM on Apr. 27, 2022.

The term “Corynebacterium accolens SU006” refers to the Corynebacterium accolens SU006 strain having the accession number NCAIM P (B) 001502 deposited at NCAIM on Apr. 27, 2022.

The term “Corynebacterium propinquum SU007” refers to the Corynebacterium propinquum SU007 strain having the accession number NCAIM P (B) 001504 deposited at NCAIM on Apr. 27, 2022.

The term “Corynebacterium accolens SU008” refers to the Corynebacterium accolens SU008 strain having the accession number NCAIM P (B) 001505 deposited at NCAIM on Apr. 27, 2022.

The term “Corynebacterium accolens SU009” refers to the Corynebacterium accolens SU009 strain having the accession number NCAIM P (B) 001506 deposited at NCAIM on May 25, 2022.

The term “Corynebacterium propinquum SU010” refers to the Corynebacterium propinquum SU010 strain having the accession number NCAIM P (B) 001507 deposited at NCAIM on May 25, 2022.

### BRIEF DESCRIPTION OF THE FIGURES

FIG. 1. The temperatures in the lyophilization process.

FIG. 2A. The effect on the relative mRNA expression of cathepsin of the different Corynebacterium strains (SU001-SU010).

FIG. 2B. The effect on the relative mRNA expression of cathepsin of the different Corynebacterium strains (SU004-SU010).

FIG. 3. The effect on the relative mRNA expression of cathepsin, ACE2 and TMPRSS2 of the different Corynebacterium strains (SU001-SU010).

FIG. 4. The effect on the relative mRNA expression of cathepsin, ACE2 and TMPRSS2 of the different Corynebacterium strains (SU004-SU010).

FIG. 5. The effect on ACE2-S1 protein binding activity (%) of the different Corynebacterium strains (SU001-SU010).

FIG. 6. The effect on ACE2-S1 protein binding activity (%) of the different Corynebacterium strains (SU004-SU010).

FIG. 7. The effect of K1 combination on the relative expression of Cathepsin, ACE2 and TMPRSS2.

FIG. 8. The effect of K2 combination on the relative expression of Cathepsin, ACE2 and TMPRSS2.

FIG. 9. The effect of K3 combination on the relative expression of Cathepsin, ACE2 and TMPRSS2.

FIG. 10. The effect of xanthan on the ACE2, TMPRSS2 and cathepsin expression of K1, K2 and K3 combinations.

FIG. 11. The effect of carrageenan on the ACE2, TMPRSS2 and cathepsin expression of K1, K2 and K3 combinations.

### DETAILED DESCRIPTION OF THE INVENTION

The present inventors determined that the normal flora of the nasopharynx influences susceptibility to SARS-CoV-2 infection.

The presence of Corynebacterium spp. in the nasopharyngeal flora can reduce the individual susceptibility to SARS-CoV-2 infection by several mechanisms.

Corynebacterium strains can reduce the individual susceptibility to SARS-CoV-2 infections through several mechanisms: through downregulation of SARS-CoV-2 receptors: ACE2 and TMPRSS2, through the inhibition of S1 protein and ACE2 receptor binding and through lipase production. All these mechanisms together act by inhibiting the binding of SARS-CoV-2 to the host cell and by acting on the lipid envelope of the SARS-CoV-2 virus. These mechanisms can reduce the individual susceptibility to the infection against enveloped respiratory viral infection, among others especially SARS-CoV-2.

The present inventors' aims were the following. Their goal was to determine the effect of Corynebacterium strains on the Cathepsin pathway brought to the fore by the omicron variant on human cells. After examining the effects of Corynebacterium, they deposited several strains. Their goal was to determine the ideal combination of Corynebacterium strains and determine their preventive effect against SARS-CoV-2 infection for the purpose of probiotic use, as well. Furthermore, their aim was to determine the appropriate carrier for the Corynebacterium strains for probiotic use, in addition to which the Corynebacterium strains retain their effectiveness against SARS-CoV-2 and their viability.

Given that the entry of the SARS-CoV-2 virus into the cell is not only through ACE2 and TMPRSS2, but the cathepsin pathway is becoming increasingly important, especially in the case of omicron variants, the present inventors isolated Corynebacterium strains, and examined the effect of bacterial cocultivation on cathepsin expression. The isolated Corynebacterium strains significantly reduced the relative expression of cathepsin mRNA to varying degrees after 24-hour incubation with Caco-2 cells, thereby contributing to the ACE2 expression reduction, TMPRSS2 expression reduction, ACE2-S protein inhibition, and the presence of lipase, reducing the entry of SARS-CoV-2 virus, preferably SARS-CoV-2 omicron mutant into the cells.

The present inventors proved that Corynebacteria are capable of downregulating Cathepsin protein expression on human cells. The cathepsins pathway is an important entry gate for the SARS-CoV-2 virus, and the currently widespread omicron variant primarily enters via the cathepsins pathway.

Among the Corynebacterium they investigated, ten Corynebacterium strains have been deposited under the names SU001-SU010. When depositing the Corynebacterium strains, they considered their effect on Cathepsin expression in addition to the other effects against SARS-CoV-2 infection.

Combinations K1, K2, K3 of the deposited Corynebacterium strains containing four Corynebacterium strains were determined, which, in addition to the unique beneficial properties of the Corynebacterium strains, further enhance the effect on the downregulation of the expression of ACE2, TMPRSS2 and Cathepsin.

The present inventors described that the carrier xanthan did not affect the germ count of the Corynebacterium combination K1, K2, K3 after lyophilization.

They also described that the combinations of Corynebacterium K1, K2, K3 with xanthan carrier retained their viability and germ count for 14 days at room temperature after lyophilization. Furthermore, they described that the combinations of Corynebacterium K1, K2, K3 with the carrier xanthan retained their effect on the downregulation of ACE2, TMPRSS2 and Cathepsin after lyophilization, and furthermore, it could even synergistically improve it.

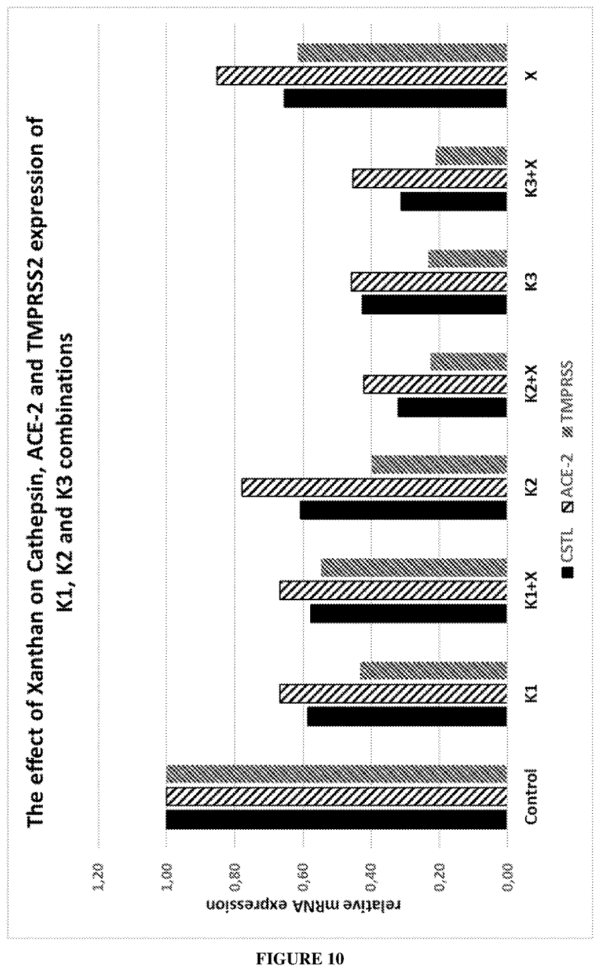

Thus, the present inventors surprisingly found that xanthan not only acted as an excipient (carrier), but as an adjuvant as well—it improved the effect of the Corynebacterium combinations, especially in combination K2 (see FIG. 10).

They described that the carrier kappa carrageenan did not affect the germ count of the Corynebacterium combination K1, K2, K3 after lyophilization. Furthermore, they described that the combinations of Corynebacterium K1, K2, K3 with kappa carrageenan carrier retained their viability and germ count for 14 days at room temperature after lyophilization. They also described that the combinations of Corynebacterium K1, K2, K3 with the carrier kappa carrageenan retained their effect on the downregulation of ACE2, TMPRSS2 and Cathepsin after lyophilization.

The combinations of bacterial strains can be reconstituted in the form of a nasal or pharyngeal spray after freeze-drying. All of them retain their known and tested biological activity.

Not just finding the appropriate excipients to be used in the lyophilized formulation, but also finding the suitable concentrations of the excipients was an important step.

The carbohydrate or carbohydrate derivative excipient in the lyophilized formulation is a cryoprotectant, it protects the Corynebacterium culture.

The concentration of the hydrocolloid polymer in the lyophilized formulation controls the viscosity of the formulation. The lyophilized formulation should not gel but should be able to form enough cakes. In other words, the formulation should not be too viscous or too dense. Too high a concentration caused gelation, and too little a concentration caused the formulation not to form appropriate cake during lyophilization.

The lyophilized formulation should have an ideal range of osmolality, as well, preferably in the range of 290 to 500 mOsmol/kg, more preferably 300 to 400 mOsm/kg. More preferably, the osmolality of the lyophilized formulation is 320 to 370 mOsmol/kg.

Preferably, the pH of the lyophilized formulation is pH 5 to 7, more preferably pH 5.5 to 6.5.

Another important aspect is that the lyophilized formulation must be physiologically tolerable, thus the excipients used and their concentration should be selected carefully.

In case of a nasal spray, droplet size distribution is also important [Kulkarni & Shaw (2012). Formulation and characterization of nasal sprays. Inhalation Magazine, June 2012]. Larger droplets will drip out of the nose, while droplets smaller than 10 μm may travel further into the nasal cavity and reach the lungs (which is not the intended delivery site). Therefore, droplets smaller than 10 μm must be kept at a minimum. For example, in a nasal spray, the size of the droplets may be 30-70 μm, at most 200 μm.

### EXAMPLES

### Example 1—Corynebacterium Strains

### Materials and Methods

### Bacterial Sample Collection

Corynebacterium isolates with a presumed protective effect were collected from patients who did not become infected, although they had close contacts with their COVID positive family members. Nasopharyngeal swab samples of the COVID negative patients were inoculated on Columbia blood agar (Biolab, Hungary), and incubated at 37 °C in a humidified atmosphere containing 5% CO₂. Antimicrobial susceptibility disc containing 50 μg Fosfomycin (Oxoid, Sweden) was used to select Corynebacterium strains from other bacterial participants. All cultured bacteria were identified by the MALDI-TOF method (Bruker Daltonik, Germany). The favourable properties of three previously isolated Corynebacterium strains (SU001-003) were examined, and another 7 Corynebacterium strains (SU004-010) were isolated and biologically tested.

Examination of ACE2, TMPRSS2 and Cathepsin L expression of Caco-2 cells after bacterial co-culture Human colonic epithelial cell line Caco-2 was previously obtained and used for this study. The Caco-2 cells were cultured in EMEM medium (Lonza Bioscience, USA) at 37 °C in a humidified atmosphere containing 5% CO₂ and the medium was changed every two days. No antibiotics were added to allow the co-cultivation of bacteria. The Caco-2 cells were cultivated alone for four days before the addition of the overnight cultures of Corynebacteria SU001-010. Human Caco-2 cells and bacteria were co-cultured for additional 24 hours.

The cells were washed by PBS and 0.25% trypsin and centrifuged. The total RNA was isolated by innuPREP RNA Mini Kit 2.0 (Analytik Jena GmbH, Germany) according to manufacturer's instructions. RNA concentrations were determined using a NanoDrop 1000 spectrophotometer (Thermo Fisher Scientific, USA). 10-100 ng of RNA was used for RT-PCR assay performed using the PrimeScript RT reagent kit (Takara Bio, USA) and amplified the resulting cDNA on a qTOWER 3G (Analytik Jena GmbH, Germany) instrument in the presence of selected primers.

The primers for ACE2 were 5′-GGG ATC AGA GAT CGG AAG AAG AAA-3′ forward (SEQ ID NO: 1) and 5′-AGG AGG TCT GAA CAT CAT CAG TG-3′ reverse (SEQ ID NO: 2). The primers for TMPRSS2 were 5′-AAT CGG TGT GTT CGC CTC TAC-3′ forward (SEQ ID NO: 3) and 5′-CGT AGT TCT CGT TCC AGT CGT-3′ reverse (SEQ ID NO: 4). The primers for Cathepsin L were 5′-CTG GTG GTT GGC TAC GGA TT-3′ CTSL forward (SEQ ID NO: 5) and 5′-CTC CGG TCT TTG GCC ATC TT-3′ reverse (SEQ ID NO: 6). The primers for GAPDH were 5′-CTA CTG GCG CTG GCA AGG CTG T-3′ forward (SEQ ID NO: 7), and 5′-GCC ATG AGG TCC ACC ACC CTG CTG-3′ reverse (SEQ ID NO: 8).

Relative mRNA expression was calculated by means of the change in cycle threshold (ΔΔCt) method and normalized to the geometric mean of the housekeeping genes GAPDH. Basal mRNA levels of ACE2, TMPRSS2 and Cathepsin were compared with those of the housekeeping gene GAPDH by calculating the difference between their Ct.

### Statistical Analysis

In ACE2, TMPRSS2 and Cathepsin expression studies, the difference between mRNA levels measured in the different bacterial co-culture groups was calculated by two-tailed students t-test.

### The Effect of Corynebacteria on ACE2, TMPRSS2 and Cathepsin Expression

In the case of the SU001-010 strains, the present inventors investigated how they affect the relative ACE2, TMPRSS2 and Cathepsin expression of the Caco-2 cells cultured together with each strain individually.

### Results

### The Effect of Corynebacterium on Cathepsin Expression

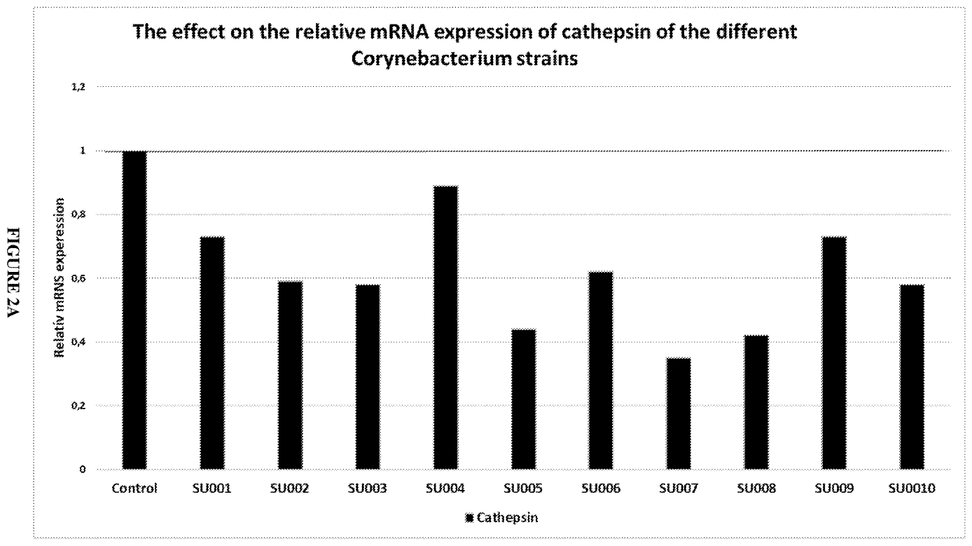

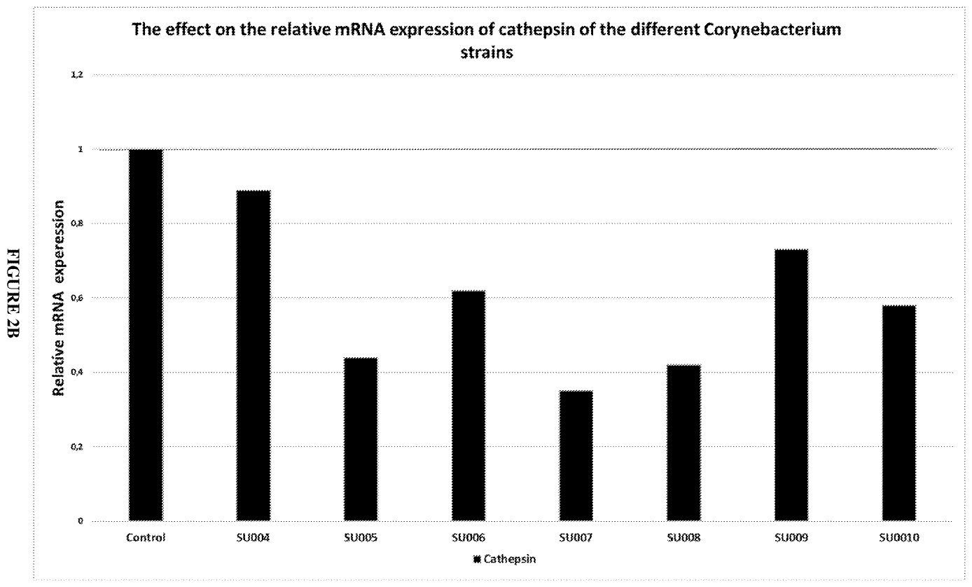

Given that the entry of the SARS-CoV-2 virus into the cell is not only through ACE2 and TMPRSS2, but the cathepsins pathway is becoming increasingly important, especially in the case of omicron variants, so in the case of the previously isolated Corynebacterium strains, we examined the effect of bacterial cocultivation on cathepsin expression. The Corynebacterium strains significantly reduced the relative expression of cathepsin mRNA to varying degrees after 24-hour incubation with Caco-2 cells (see FIGS. 2A and 2B), thereby contributing to the ACE2 expression reduction, TMPRSS2 expression reduction, ACE2-S protein inhibition, and the presence of lipase in addition to the previously described effects of the SARS-CoV-2 virus reducing the entry of omicron mutant into the cells.

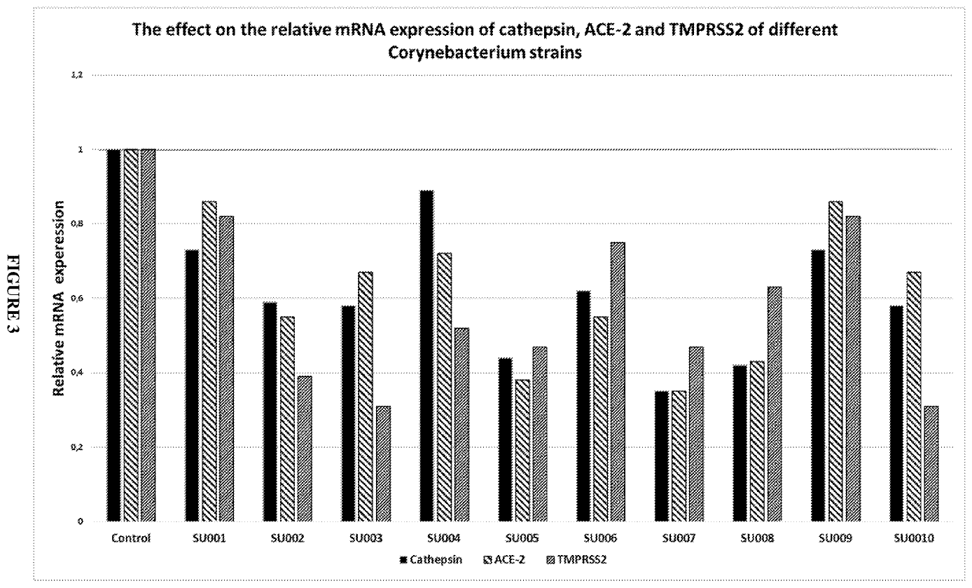

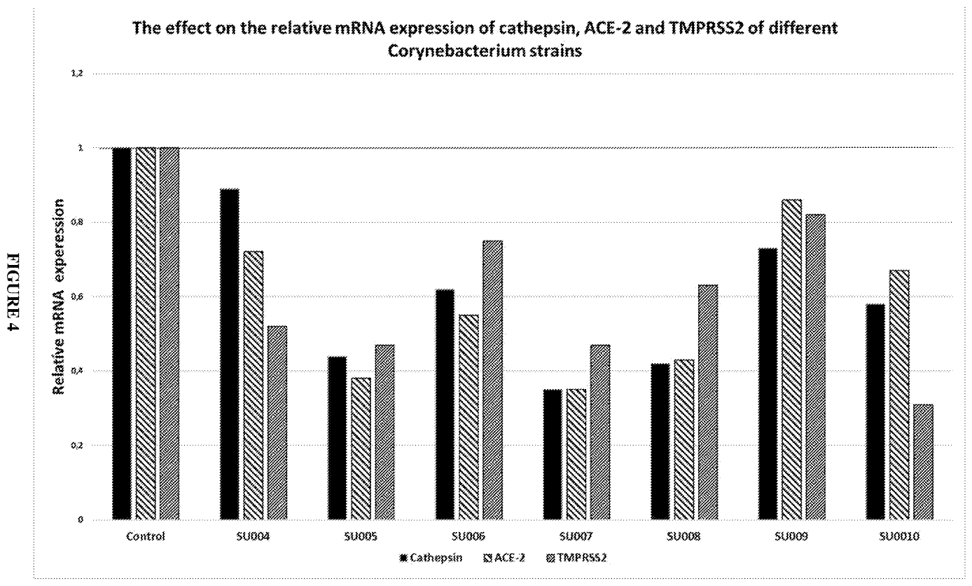

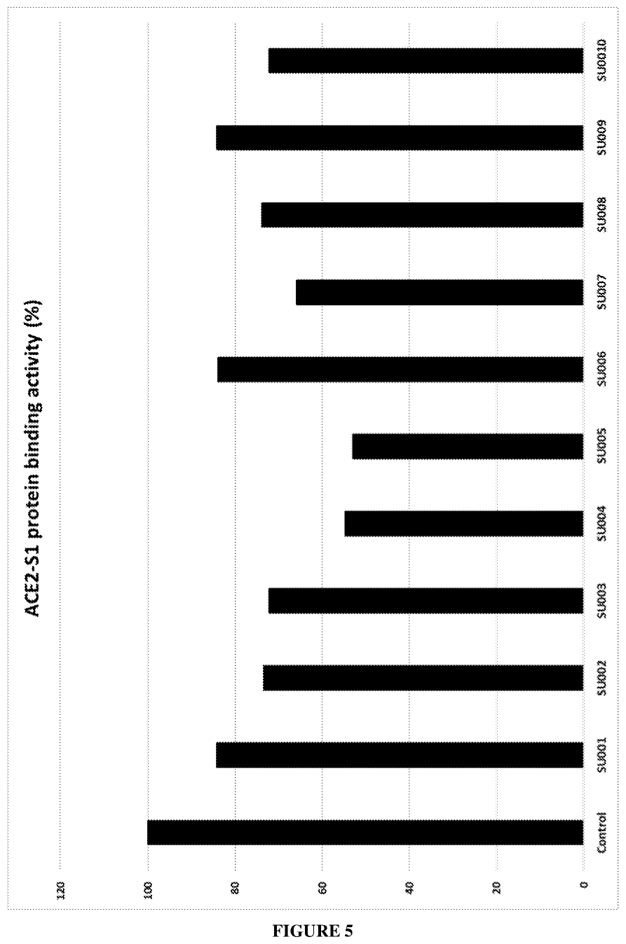

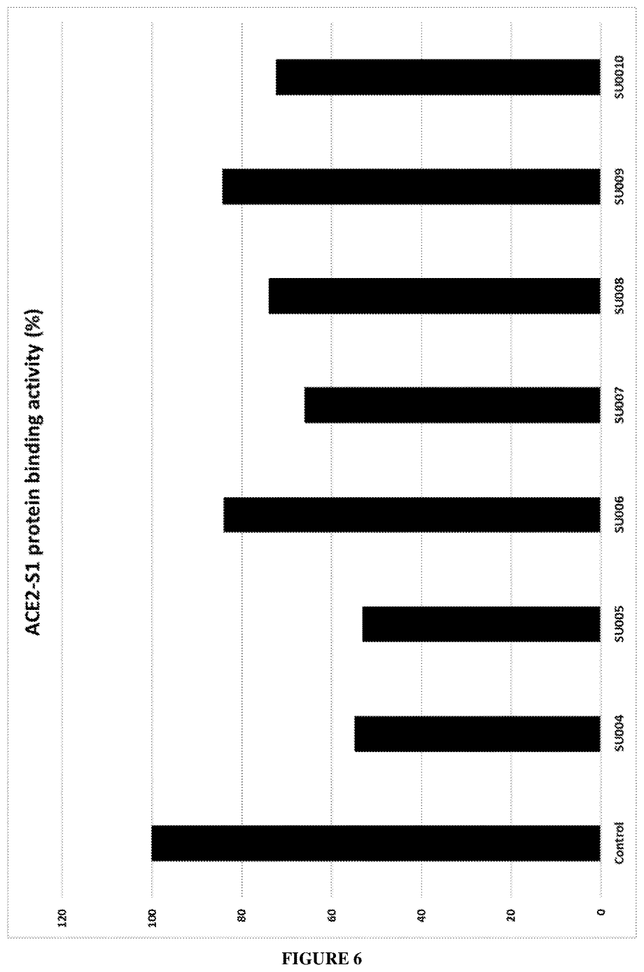

The Corynebacterium strains also reduced the relative mRNA expression of ACE2 and TMPRSS2 (see FIG. 3 and FIG. 4). Furthermore, the Corynebacterium strains inhibited the binding of S1 protein and ACE2 receptor (see FIG. 5 and FIG. 6).

### Example 2—Combinations of Corynebacterium Strains

### Materials and Methods

Culture Studies with Corynebacterium Strain Combinations

The present inventors examined how each Corynebacterium strain mutually influence each other's reproduction. A pure bacterial culture from each bacterial strain was suspended in saline, its turbidity was standardized at 0.5 McFarland, and mixtures of the same ratio were composed, which contained 4-4 strains of the SU001-010 strains in different groupings. 100 μL of the mixtures were inoculated on Columbia blood agar (Biolab, Hungary) and incubated at 37 °C in a humidified atmosphere containing 5% CO₂ for 48 hours. From the cultures, it was possible to determine which Corynebacterium strains allowed the other strains combined with them to grow, based on the different colony morphological growth. K1-K2-K3, three different combinations could be compiled based on successful joint growth.

Examination of ACE2, TMPRSS2 and Cathepsin L Expression of Caco-2 Cells after Bacterial Co-Culture

Human colonic epithelial cell line Caco-2 was previously obtained and used for this study. The Caco-2 cells were cultured in EMEM medium (Lonza Bioscience, USA) at 37 °C in a humidified atmosphere containing 5% CO₂ and the medium was changed every two days. No antibiotics were added to allow the co-cultivation of bacteria. The Caco-2 cells were cultivated alone for four days before the addition of the overnight cultures of K1, K2 or K3 Corynebacterium strains combination. Human Caco-2 cells and bacteria were co-cultured for additional 24 hours.

The cells were washed by PBS and 0.25% trypsin and centrifuged. The total RNA was isolated by innuPREP RNA Mini Kit 2.0 (Analytik Jena GmbH, Germany) according to manufacturer's instructions. RNA concentrations were determined using a NanoDrop 1000 spectrophotometer (Thermo Fisher Scientific, USA). 10-100 ng of RNA was used for RT-PCR assay performed using the PrimeScript RT reagent kit (Takara Bio, USA) and amplified the resulting cDNA on a qTOWER 3G (Analytik Jena GmbH, Germany) instrument in the presence of selected primers.

The primers for ACE2 were 5′-GGG ATC AGA GAT CGG AAG AAG AAA-3′ forward (SEQ ID NO: 1) and 5′-AGG AGG TCT GAA CAT CAT CAG TG-3′ reverse (SEQ ID NO: 2). The primers for TMPRSS2 were 5′-AAT CGG TGT GTT CGC CTC TAC-3′ forward (SEQ ID NO: 3) and 5′-CGT AGT TCT CGT TCC AGT CGT-3′ reverse (SEQ ID NO: 4). The primers for Cathepsin L were 5′-CTG GTG GTT GGC TAC GGA TT-3′ CTSL forward (SEQ ID NO: 5) and 5′-CTC CGG TCT TTG GCC ATC TT-3′ reverse (SEQ ID NO: 6). The primers for GAPDH were 5′-CTA CTG GCG CTG GCA AGG CTG T-3′ forward (SEQ ID NO: 7), and 5′-GCC ATG AGG TCC ACC ACC CTG CTG-3′ reverse (SEQ ID NO: 8).

Relative mRNA expression was calculated by means of the change in cycle threshold (ΔΔCt) method and normalized to the geometric mean of the housekeeping genes GAPDH. Basal mRNA levels of ACE2, TMPRSS2 and Cathepsin were compared with those of the housekeeping gene GAPDH by calculating the difference between their Ct.

### Statistical Analysis

In ACE2, TMPRSS2 and Cathepsin expression studies, the difference between mRNA levels measured in the different bacterial co-culture groups was calculated by two-tailed students t-test.

The Effect of Different Corynebacteria Strains Combinations (K1, K2, K3) on their Individual Biological Effects: ACE2 Expression, TMPRSS2 Expression, Cathepsin Expression

In the case of the K1, K2 and K3 combinations, the present inventors investigated how they affect the relative ACE2, TMPRSS2 and Cathepsin expression of the Caco-2 cells cultured together with each combination individually.

### Results

The Effect of Different Corynebacteria Strains Combinations (K1, K2, K3) on their Individual Biological Effects: ACE2 Expression, TMPRSS2 Expression, Cathepsin Expression

In the course of the present inventors' studies, in order to examine the effect of the Corynebacteria together on the beneficial properties of the strains previously examined individually, they cultured four-four strains together and examined their combined effect.

The following strain combinations were used: (i) combination K1 comprising Corynebacterium strains SU004, SU005, SU009, and SU010; (ii) combination K2 comprising Corynebacterium strains SU004, SU005, SU006, and SU010; and (iii) combination K3 comprising Corynebacterium strains SU004, SU005, SU007, and SU008.

In the case of all three combinations, their results showed that the different Corynebacterium strains used in the combination did not influence each other's individual effects during the co-cultivation, and surprisingly, in some cases the effect enhancing the beneficial outcome was observed.

The Effect of K1 Combination on mRNA Expression of Cathepsin, ACE2 and TMPRSS2

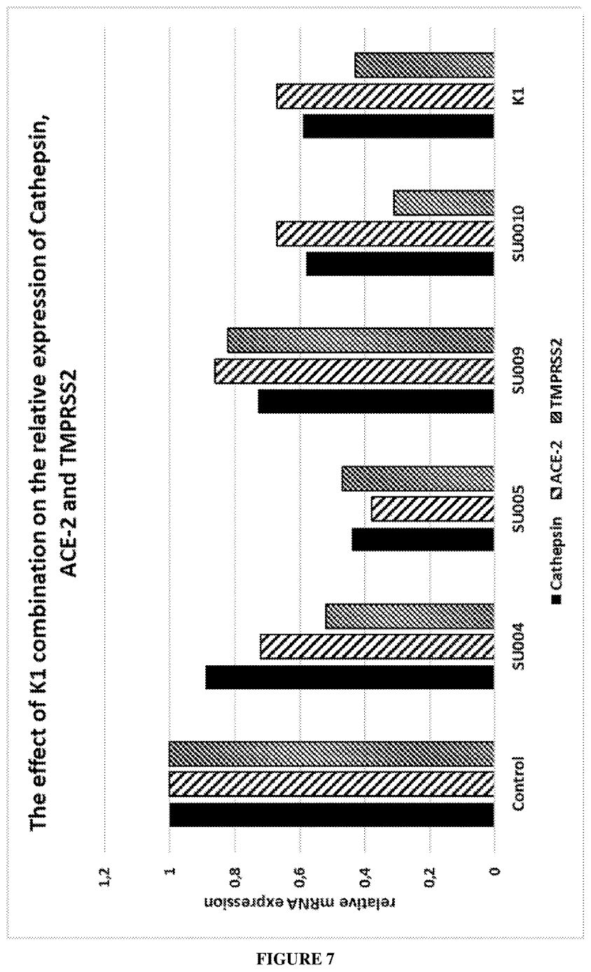

The K1 combination contained two C. accolens strains SU004, SU009, one C. tuberculostearicum SU005 and one C. propinquum SU010. When using the K1 combination, the effect of the Corynebacterium combination on Cathepsin, ACE2 and TMPRSS2 expression is approximately the average of the individual effects of each bacterium (see FIG. 7).

The Effect of K2 Combination on mRNA Expression of Cathepsin, ACE2 and TMPRSS2

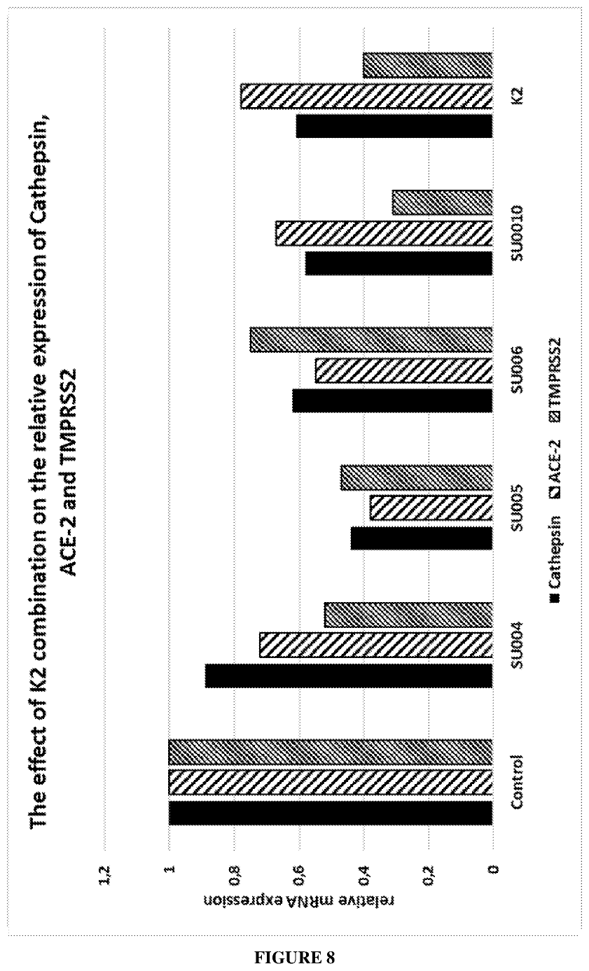

The K2 combination contained two C. accolens strains SU004, SU006, one C. tuberculostearicum SU005 and one C. propinquum SU010. When using the K2 combination, the effect of the Corynebacterium combination on Cathepsin and TMPRSS2 expression is approximately the average of the individual effects of each bacterium and in case of ACE2 the relative expression rate is similar to SU004 strain (see FIG. 8).

The Effect of K3 Combination on mRNA Expression of Cathepsin, ACE2 and TMPRSS2

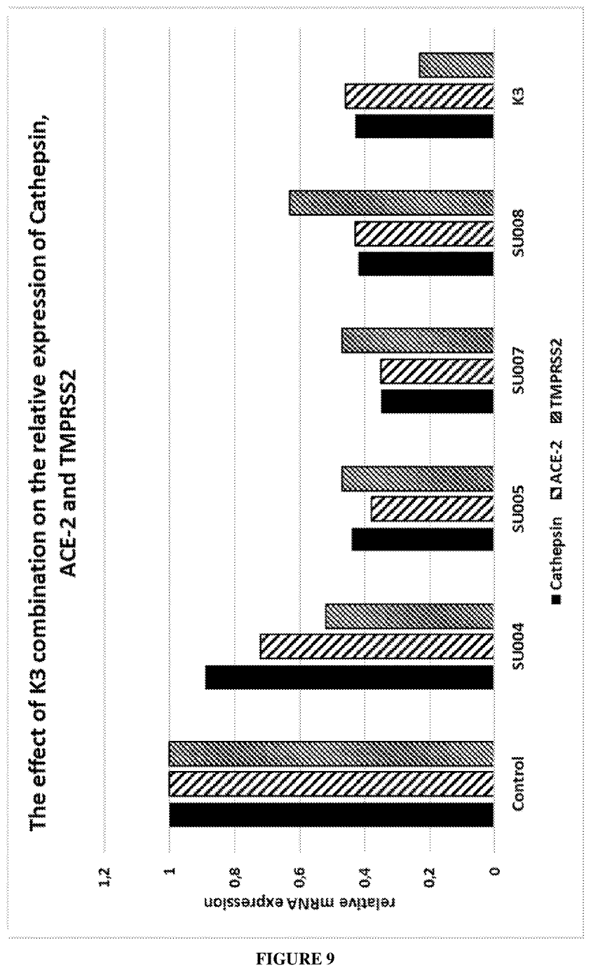

The K3 combination contained two C. accolens strains SU004, SU008, one C. tuberculostearicum SU005 and one C. propinquum SU007. When using the K3 combination, the effect of the Corynebacterium combination on Cathepsin and ACE2 expression is very similar to the values of the most effective strains, however in case of TMPRSS2 expression the K3 combination has even more effect than each bacterium alone, it is synergistic in case of TMPRSS2 expression downregulation (see FIG. 9).

### Example 3—Lyophilized Formulations

### Materials and Methods

### Investigation of Framework-Forming Properties of Different Hydrocolloid Polymer and Carbohydrate-Containing Solutions for Lyophilization

The following excipients and excipient combinations were examined: (1) 0.05 g xanthan/100 mL and vitamin A; (2) 0.1 g/100 mL kappa carrageenan and 0.5 g/100 mL maltodextrin and vitamin A; (3) 0.1 g/100 mL kappa carrageenan and 0.1 g/100 mL maltodextrin and vitamin A; (4) 0.05 g xanthan/100 mL and 0.1 g/100 mL maltodextrin and vitamin A; (5) 0.05 g xanthan/100 mL and 0.5 g/100 mL maltodextrin and vitamin A; (6) (also called A solution) 0.1 g/100 mL kappa carrageenan and 0.5 g/100 mL isomaltulose; (7) (also called B solution) 0.05 g xanthan/100 mL and 0.5 g/100 mL isomaltulose.

Early experiments showed that vitamin A reduces the effect of the excipient combination. Thus, the present inventors excluded vitamin A from the excipient combination, and they selected combinations (6) and (7) (also called A solution and B solution, respectively) for their later experiments.

### Lyophilization

One of the aims of the present inventors was to compound a nasal spray probiotic and to test the viability of bacterial strains after freeze-drying and storage during a period of two weeks.

The stabilization of bacterial strains in dry solid state were performed by a freeze dyer (Scanvac Coolsafe 110-04 cryosiccator LaboGene™, Lynge, Denmark).

The lyophilization process usually consists of three steps, namely freezing, primary drying and secondary drying. During freezing, the water is crystallized, then the frozen ice is removed by sublimation. In the final secondary drying step, the remaining water is removed reaching the final moisture content.

As cryoprotectants hydrocolloid polymers and carbohydrates were used for lyophilisation of probiotics with satisfactory survival rate of the microorganisms after the process. Generally, carbohydrates (e.g. trehalose, sucrose, glucose, lactose, maltodextrin) and polymers are applied as cryoprotectans. [Meng X C, Stanton C, Fitzgerald G F, Daly C, Ross R P. (2008). Anhydrobiotics: The challenges of drying probiotic cultures. Food Chem., 106:1406-1416.]

It is technologically important to combine these cryoprotectant compounds to ensure the survival rate of bacteria with a suitable composition for lyophilisation and characterized by a stable physical form which can be reconstituted as sprayable liquid with proper viscosity, pH (5-7) and osmolality (300-400 mosm). Preferably, the pH of a suitable composition is pH 5.5 to 6.5, and the osmolality of a suitable composition is 320 to 370 mOsmol/kg.

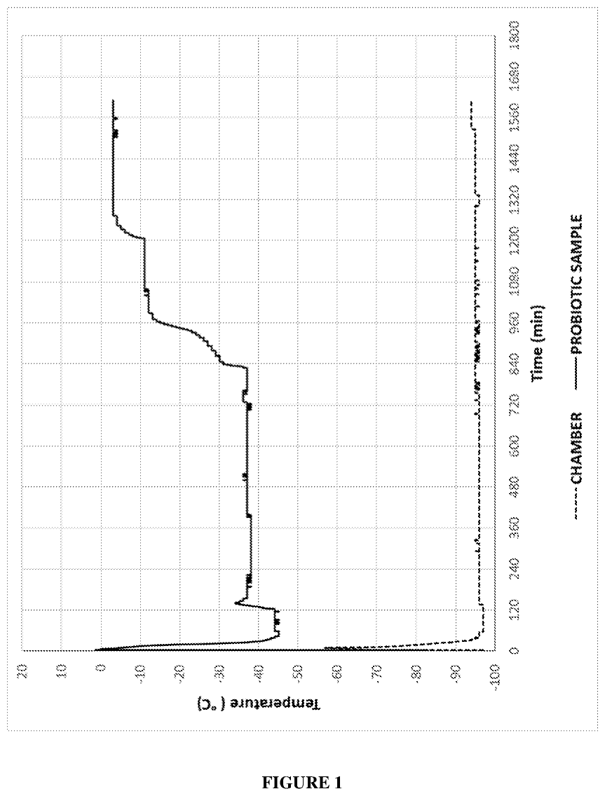

In the formation of the lyophilisate, hydrocolloid polymers and carbohydrate derivatives were used as protectants. 10⁸ CFU/mL 2 mL Ringer solution from each bacterial strain was frozen at −70 °C and stored for 24 hours. The lyophilization started with the first drying between −30 °C and −45 °C for not more than 16 hours. The secondary drying was performed until a suitable moisture content of not more than 5% maintaining the sample temperature not exceeding 10 °C temperature. The entire lyophilization process took 24-36 hours. [Zayed, G. and Y. H. Roos. 2004. Influence of trehalose and moisture content on survival of Lactobacillus salivarius subjected to freeze-drying and storage. Process Biochem. 9: 1081-1086.] See also FIG. 1.

### Shelf-Life Studies in Different Hydrocolloid Polymer and Carbohydrate Containing Solutions

Two different solutions were prepared in Ringer solution containing: (A) 0.1 g/100 mL kappa carrageenan and 0.5 g/100 mL isomaltulose (A solution), or (B) 0.05 g xanthan/100 mL and 0.5 g/100 mL isomaltulose (B solution).

Portions of 2 mL of A or B solutions were inoculated with 10⁸ CFU (Colony Forming Unit)/mL SU001-010 strains or with K1, K2, K3 strain combinations. After 7 and 14 days of storage at room temperature, 100 μL was inoculated from the different solution onto Columbia blood agar (Biolab, Hungary) to determine the number of Colony Forming Units as the amount of viable Corynebacterium strains.

Examination of ACE2, TMPRSS2 and Cathepsin L Expression of Caco-2 Cells after Bacterial Co-Culture

Human colonic epithelial cell line Caco-2 was previously obtained and used for this study. The Caco-2 cells were cultured in EMEM medium (Lonza Bioscience, USA) at 37 °C in a humidified atmosphere containing 5% CO₂ and the medium was changed every two days. No antibiotics were added to allow the co-cultivation of bacteria. The Caco-2 cells were cultivated alone for four days before the addition of the overnight cultures of Corynebacteria SU001-010, or K1, K2 or K3 combination in A or B solution. Human Caco-2 cells and bacteria were co-cultured for additional 24 hours.

The cells were washed by PBS and 0.25% trypsin and centrifuged. The total RNA was isolated by innuPREP RNA Mini Kit 2.0 (Analytik Jena GmbH, Germany) according to manufacturer's instructions. RNA concentrations were determined using a NanoDrop 1000 spectrophotometer (Thermo Fisher Scientific, USA). 10-100 ng of RNA was used for RT-PCR assay performed using the PrimeScript RT reagent kit (Takara Bio, USA) and amplified the resulting cDNA on a qTOWER 3G (Analytik Jena GmbH, Germany) instrument in the presence of selected primers.

The primers for ACE2 were 5′-GGG ATC AGA GAT CGG AAG AAG AAA-3′ forward (SEQ ID NO: 1) and 5′-AGG AGG TCT GAA CAT CAT CAG TG-3′ reverse (SEQ ID NO: 2). The primers for TMPRSS2 were 5′-AAT CGG TGT GTT CGC CTC TAC-3′ forward (SEQ ID NO: 3) and 5′-CGT AGT TCT CGT TCC AGT CGT-3′ reverse (SEQ ID NO: 4). The primers for Cathepsin L were 5′-CTG GTG GTT GGC TAC GGA TT-3′ CTSL forward (SEQ ID NO: 5) and 5′-CTC CGG TCT TTG GCC ATC TT-3′ reverse (SEQ ID NO: 6). The primers for GAPDH were 5′-CTA CTG GCG CTG GCA AGG CTG T-3′ forward (SEQ ID NO: 7), and 5′-GCC ATG AGG TCC ACC ACC CTG CTG-3′ reverse (SEQ ID NO: 8).

Relative mRNA expression was calculated by means of the change in cycle threshold (ΔΔCt) method and normalized to the geometric mean of the housekeeping genes GAPDH. Basal mRNA levels of ACE2, TMPRSS2 and Cathepsin were compared with those of the housekeeping gene GAPDH by calculating the difference between their Ct.

### Statistical Analysis

In ACE2, TMPRSS2 and Cathepsin expression studies, the difference between mRNA levels measured in the different bacterial co-culture groups was calculated by two-tailed students t-test.

### The Effect of Different Hydrocolloid Polymer and Carbohydrate Containing Solutions on the Individual Biological Effects of SU001-010 Strains or K1-K3 Compositions on ACE2 Expression, TMPRSS2 Expression and Cathepsin Expression

In the case of the SU001-SU010 strains, or K1, K2 and K3 combinations in A or B solutions, the present inventors investigated how they affect the relative ACE2, TMPRSS2 and Cathepsin expression of the Caco-2 cells cultured together with each strain or combination individually.

### Results

### The Effect of Different Excipient (Xanthan and Kappa Carrageenan) on the Corynebacterium Strains Combinations K1, K2, K3

The various combinations K1, K2 and K3 found to be effective were studied together with different excipient in order to determine the effect of the excipient on the combinations of bacteria. The present inventors further investigated the effect of two excipient, the xanthan, an anionic polysaccharide and the kappa carrageenan on the biological activity of the Corynebacterium strains.

### Investigation of the Effect of Lyophilization on the Growth of Corynebacteria

The present inventors further examined the effect of lyophilization on different the Corynebacterium strains individually and on the combinations K1, K2 and K3. After lyophilization, the present inventors determined the germ count of each bacterium separately and also combinations K1, K2, K3. Their results showed that all bacteria and combinations kept their original amount of bacteria.

TABLE 1
The effect of lyophilization
The effect of lyophilization

| Strains | kappa carrageenan | xanthan |
|---|---|---|
| SU001 | 10⁷ CFU/mL | 10⁸ CFU/mL |
| SU002 | 10⁶ CFU/mL | 10⁸ CFU/mL |
| SU003 | 10⁴ CFU/mL | 10⁸ CFU/mL |
| SU004 | 10⁷ CFU/mL | 10⁸ CFU/mL |
| SU005 | 10³ CFU/mL | 10⁸ CFU/mL |
| SU006 | 10⁵ CFU/mL | 10⁸ CFU/mL |
| SU007 | 10⁷ CFU/mL | 10⁸ CFU/mL |
| SU008 | 10⁸ CFU/mL | 10⁸ CFU/mL |
| SU009 | 10⁷ CFU/mL | 10⁸ CFU/mL |
| SU010 | 10⁴ CFU/mL | 10⁸ CFU/mL |
| K1 (SU004, SU005, SU009, SU010) | 10⁵ CFU/mL | 10⁸ CFU/mL |
| K2 (SU004, SU005, SU006, SU010) | 10⁶ CFU/mL | 10⁹ CFU/mL |
| K3 (SU004, SU005, SU007, SU008) | 10⁶ CFU/mL | 10⁹ CFU/mL |

They further analysed the survival of the Corynebacterium strain and in all combinations K1, K2 and K3 Corynebacterium strains could survive for 14 days.

The Effect of Xanthan on mRNA Expression of Cathepsin, ACE2 and TMPRSS2

The present inventors further analysed the effect of the xanthan alone and in the mixture with K1, K2 and K3 combinations for their effect on the relative expression of ACE2, TMPRSS2 and Cathepsin relative expression. The xanthan alone itself decreased the expression of the ACE2, TMPRSS2 and Cathepsin, however, to a lesser extent than K1, K2 and K3 combination.

Interestingly, K1 and K3 in combination with xanthan did not affect their original effect, however, surprisingly, in the case of the K2 combination, a synergistic effect was observed with xanthan, as the combined presence of K2 and xanthan downregulated the ACE2, TMPRSS2 and Cathepsin expression to a greater extent than separately (see FIG. 10).

The Effect of Kappa-Carrageenan on mRNA Expression of Cathepsin, ACE2 and TMPRSS2

They further analysed the effect of the kappa-carrageenan alone and in the mixture with K1, K2 and K3 combinations for their effect on the relative expression of ACE2, TMPRSS2 and Cathepsin relative expression. The kappa carrageenan alone itself also decreased significantly the expression of the ACE2, TMPRSS2 and Cathepsin.

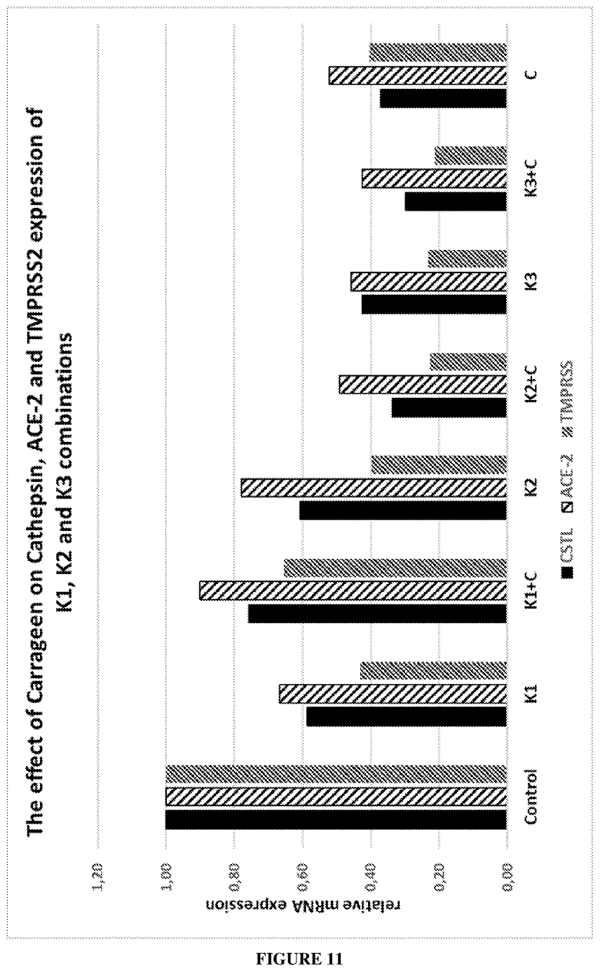

However, interestingly, in case of K1 combination with the kappa-carrageenan had no additional effect. Surprisingly, in case of K2 and kappa-carrageenan, synergistic effect was observed, all the ACE2, TMPRSS2 and Cathepsin expression was downregulated to a much greater degree when using K2 and kappa-carrageenan together. In case of K3, the kappa-carrageenan did not influence the K3 combination caused downregulation effect of ACE2, TMPRSS2 and Cathepsin expression (see FIG. 11).

### INDUSTRIAL APPLICABILITY

The Corynebacterium culture or combination or lyophilized formulation thereof according to the invention is for use in the treatment or prevention of a respiratory disease of the upper respiratory tract. The Corynebacterium culture or combination or lyophilized formulation according to the invention is also for use in the treatment or prevention of a respiratory tract infection, respiratory tract colonization or respiratory tract disease. Preferably, the respiratory tract infection, the respiratory tract colonization or the respiratory tract disease is of the upper respiratory tract. The Corynebacterium culture or combination or lyophilized formulation according to the invention is for use in the prevention of a disease caused by an infectious agent that settles in the respiratory tract, preferably in the upper respiratory tract.

### REFERENCES

Kulkarni & Shaw (2012). Formulation and characterization of nasal sprays. Inhalation Magazine, June 2012
Lappan & Peacock, 2019. Corynebacterium and Dolosigranulum: future probiotic candidates for upper respiratory tract infections. Microbiology Australia, 40(4), 172-177.
De Maio F, Posteraro B, Ponziani F R, Cattani P et al. (2020). Nasopharyngeal Microbiota Profiling of SARS-CoV-2 Infected Patients. Biological Procedures Online 22, 18. doi: 10.1186/s12575-020-00131-7.
Man W H, de Steenhuijsen Piters W A, Bogaert D (2017). The microbiota of the respiratory tract: Gatekeeper to respiratory health. Nature Reviews Microbiology, 15(5), 259-270. doi: 10.1038/nrmicro.2017.14.
Meng X C, Stanton C, Fitzgerald G F, Daly C, Ross R P. (2008). Anhydrobiotics: The challenges of drying probiotic cultures. Food Chem., 106:1406-1416.
Mostafa H H, Fissel J A, Fanelli B, Bergman Y et al. (2020). Metagenomic next-generation sequencing of nasopharyngeal specimens collected from confirmed and suspect COVID-19 patients. MBio, 11(6), 1-13. doi: 10.1128/mBio.01969-20.
Nardelli C, Gentile I, Setaro M, Di Domenico C, et al. (2021). Nasopharyngeal Microbiome Signature in COVID-19 Positive Patients: Can We Definitively Get a Role to Fusobacterium periodonticum? Frontiers in Cellular and Infection Microbiology, 11(February), 1-7. doi: 10.3389/fcimb.2021.625581.
Rosas-Salazar C, Kimura K S, Shilts M H, Strickland B A et al. (2021). SARS-CoV-2 infection and viral load are associated with the upper respiratory tract microbiome. J Allergy Clin Immunol. 2021; 147(4):1226-1233.e2. doi: 10.1016/j.jaci.2021.02.001.
Tchoupou Saha et al., 2022. Profile of the Nasopharyngeal Microbiota Affecting the Clinical Course in COVID-19 Patients. Front. Microbiol. 13:871627. doi: 10.3389/fmicb.2022.871627
Zayed, G. and Y. H. Roos. 2004. Influence of trehalose and moisture content on survival of Lactobacillus salivarius subjected to freeze-drying and storage. Process Biochem. 9: 1081-1086.

1. A method for the prevention of an infection by a coronavirus, the method comprising
administering a Corynebacterium culture capable of reducing the expression of a cathepsin to a subject in need thereof.

2. The method according to claim 1, wherein the Corynebacterium culture capable of reducing the expression of a cathepsin is a Corynebacterium culture that reduces the relative expression of cathepsin mRNA after 24-hour incubation with Caco-2 cells.

3. The method according to claim 1, wherein said coronavirus uses cathepsin pathway, preferably primarily uses cathepsin pathway for entry into the cell of the subject and
preferably wherein the Corynebacterium culture capable of reducing the expression of a cathepsin is a Corynebacterium culture that reduces the relative expression of cathepsin mRNA after 24-hour incubation with Caco-2 cells.

4. The method according to claim 1, wherein the coronavirus is SARS-CoV-2.

5. The method according to claim 1, wherein the coronavirus is SARS-CoV-2 omicron variant.

6. The method according to claim 1, wherein the Corynebacterium culture comprises at least one or at least two, preferably at least three, more preferably four Corynebacterium strain(s) selected from the group of strains of Corynebacterium accolens, Corynebacterium propinquum and Corynebacterium tuberculostearicum.

7. A method for the prevention of an infection by a SARS-CoV-2 virus, the method comprising
administering a Corynebacterium culture to a subject in need thereof,
wherein the Corynebacterium culture comprises at least one or at least two, preferably at least three, more preferably four Corynebacterium strains selected from
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16, Hungary) on Jan. 17, 2022, or a derivative, variant or mutant thereof, wherein the derivative, variant or mutant thereof is capable of reducing cathepsin expression;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression; or
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression;
preferably wherein any derivative, variant or mutant that is capable of reducing cathepsin expression is a derivative, variant or mutant, respectively that reduces the relative expression of cathepsin mRNA after 24-hour incubation with Caco-2 cells.

8. The method according to claim 1, wherein said coronavirus uses cathepsin pathway, preferably primarily uses cathepsin pathway for entry into the cell of the subject and
preferably wherein the Corynebacterium culture capable of reducing the expression of a cathepsin is a Corynebacterium culture that reduces the relative expression of cathepsin mRNA after 24-hour incubation with Caco-2 cells,
wherein preferably the coronavirus is SARS-CoV-2 virus, more preferably a SARS-CoV-2 omicron variant, and the Corynebacterium culture comprises at least one of
Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16, Hungary) on Jun. 22, 2021, or a derivative, variant or mutant thereof, wherein the derivative, variant or mutant thereof is capable of reducing cathepsin expression;
Corynebacterium propinquum SU002 strain having the accession number NCAIM P (B) 001496 deposited at NCAIM on Jun. 22, 2021, or a derivative, variant or mutant thereof, wherein the derivative, variant or mutant thereof is capable of reducing cathepsin expression;
Corynebacterium propinquum SU003 strain having the accession number NCAIM P (B) 001497 deposited at NCAIM on Jun. 22, 2021, or a derivative, variant or mutant thereof, wherein the derivative, variant or mutant thereof is capable of reducing cathepsin expression;
Corynebacterium accolens SU004 strain having the accession number NCAIM P (B) 001500 deposited at NCAIM on Jan. 17, 2022, or a derivative, variant or mutant thereof, wherein the derivative, variant or mutant thereof is capable of reducing cathepsin expression;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression; or
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression;
preferably wherein any derivative, variant or mutant that is capable of reducing cathepsin expression is a derivative, variant or mutant, respectively that reduces the relative expression of cathepsin mRNA after 24-hour incubation with Caco-2 cells.

9-15. (canceled)

16. A method for the prevention of an infection by a SARS-CoV-2 virus, the method comprising
administering a Corynebacterium strain to a subject in need thereof,
wherein the Corynebacterium strain is selected from
Corynebacterium accolens SU004 strain having the accession number NCAIM P (B) 001500 deposited at NCAIM on Jan. 17, 2022;
Corynebacterium tuberculostearicum SU005 strain having the accession number NCAIM P (B) 001501 deposited at NCAIM on Apr. 27, 2022;
Corynebacterium accolens SU006 strain having the accession number NCAIM P (B) 001502 deposited at NCAIM on Apr. 27, 2022;
Corynebacterium propinquum SU007 strain having the accession number NCAIM P (B) 001504 deposited at NCAIM on Apr. 27, 2022;
Corynebacterium accolens SU008 strain having the accession number NCAIM P (B) 001505 deposited at NCAIM on Apr. 27, 2022;
Corynebacterium accolens SU009 strain having the accession number NCAIM P (B) 001506 deposited at NCAIM on May 25, 2022; or
Corynebacterium propinquum SU010 strain having the accession number NCAIM P (B) 001507 deposited at NCAIM on May 25, 2022;
or any derivative, variant or mutant thereof capable of reducing cathepsin expression.

17. The method according to claim 1, wherein the Corynebacterium culture comprises four Corynebacterium strains selected from:
(i) Corynebacterium accolens SU004, Corynebacterium tuberculostearicum SU005, Corynebacterium accolens SU009 and Corynebacterium propinquum SU010, or any derivative, variant or mutant thereof capable of reducing cathepsin expression;
(ii) Corynebacterium accolens SU004, Corynebacterium tuberculostearicum SU005, Corynebacterium accolens SU006, and Corynebacterium propinquum SU010, or any derivative, variant or mutant thereof capable of reducing cathepsin expression; and
(iii) Corynebacterium accolens SU004, Corynebacterium tuberculostearicum SU005, Corynebacterium propinquum SU007 and Corynebacterium accolens SU008, or any derivative, variant or mutant thereof capable of reducing cathepsin expression.

18. A method for the prevention of an infection by a SARS-CoV-2 virus, the method comprising
administering a Corynebacterium culture to a subject in need thereof,
wherein the Corynebacterium culture comprises four Corynebacterium strains, which are Corynebacterium accolens SU004, Corynebacterium tuberculostearicum SU005, Corynebacterium propinquum SU007 and Corynebacterium accolens SU008.

19. A lyophilized formulation, wherein the lyophilized formulation comprises a Corynebacterium culture or strain capable of reducing the expression of a cathepsin, and a cryoprotectant.

20. The lyophilized formulation according to claim 19,
(i) wherein the coronavirus is SARS-CoV-2, and
(ii) wherein the lyophilized formulation comprises a Corynebacterium culture or strain Corynebacterium accolens SU001 strain having the accession number NCAIM P (B) 001495 deposited at NCAIM (National Collection of Agricultural and Industrial Microorganisms, Institute of Food Science and Technology, Hungarian University of Agriculture and Life Sciences, H-1118, Budapest, Somlói út 14-16, Hungary) on Jun. 22, 2021, or a derivative, variant or mutant thereof, wherein the derivative, variant or mutant thereof is capable of reducing cathepsin expression;
Corynebacterium propinquum SU002 strain having the accession number NCAIM P (B) 001496 deposited at NCAIM on Jun. 22, 2021, or a derivative, variant or mutant thereof, wherein the derivative, variant or mutant thereof is capable of reducing cathepsin expression;
Corynebacterium propinquum SU003 strain having the accession number NCAIM P (B) 001497 deposited at NCAIM on Jun. 22, 2021, or a derivative, variant or mutant thereof, wherein the derivative, variant or mutant thereof is capable of reducing cathepsin expression;
Corynebacterium accolens SU004 strain having the accession number NCAIM P (B) 001500 deposited at NCAIM (National Collection of Agricultural and Industrial Microorganisms, Institute of Food Science and Technology, Hungarian University of Agriculture and Life Sciences, H-1118, Budapest, Somlói út 14-16, Hungary) on Jan. 17, 2022, or a derivative, variant or mutant thereof, wherein the derivative, variant or mutant thereof is capable of reducing cathepsin expression;
Corynebacterium tuberculostearicum SU005 strain having the accession number NCAIM P (B) 001501 deposited at NCAIM on Apr. 27, 2022, or a derivative, variant or mutant thereof, wherein the derivative, variant or mutant thereof is capable of reducing cathepsin expression;
Corynebacterium accolens SU006 strain having the accession number NCAIM P (B) 001502 deposited at NCAIM on Apr. 27, 2022, or a derivative, variant or mutant thereof, wherein the derivative, variant or mutant thereof is capable of reducing cathepsin expression;
Corynebacterium propinquum SU007 strain having the accession number NCAIM P (B) 001504 deposited at NCAIM on Apr. 27, 2022, or a derivative, variant or mutant thereof, wherein the derivative, variant or mutant thereof is capable of reducing cathepsin expression;
Corynebacterium accolens SU008 strain having the accession number NCAIM P (B) 001505 deposited at NCAIM on Apr. 27, 2022, or a derivative, variant or mutant thereof, wherein the derivative, variant or mutant thereof is capable of reducing cathepsin expression;
Corynebacterium accolens SU009 strain having the accession number NCAIM P (B) 001506 deposited at NCAIM on May 25, 2022, or a derivative, variant or mutant thereof, wherein the derivative, variant or mutant thereof is capable of reducing cathepsin expression; or
Corynebacterium propinquum SU010 strain having the accession number NCAIM P (B) 001507 deposited at NCAIM on May 25, 2022, or a derivative, variant or mutant thereof, wherein the derivative, variant or mutant thereof is capable of reducing cathepsin expression, and
(iii) wherein the cryoprotectant comprises a hydrocolloid polymer and a carbohydrate or carbohydrate derivative,
preferably wherein the carbohydrate or carbohydrate derivative is selected from trehalose, sucrose, glucose, lactose, mannitol, sorbitol, inulin, maltodextrin and isomaltulose, preferably from maltodextrin and isomaltulose, and
preferably wherein the hydrocolloid polymer is selected from hydroxypropyl methylcellulose, hydroxy-ethyl-cellulose, carboxymethylcellulose, guar gum, carrageenan and xanthan, preferably from kappa carrageenan and xanthan.

21. The lyophilized formulation according to claim 20, wherein the carbohydrate or carbohydrate derivative is isomaltulose in a concentration of 0.5 g/100 mL to 2.5 g/100 mL, and the hydrocolloid polymer is kappa carrageenan in a concentration of 0.1 g/100 mL to 0.5 g/100 mL or xanthan in a concentration of 0.05 g/100 mL to 0.25 g/100 mL, and/or wherein the Corynebacterium culture comprises 10³ CFU/mL to 10¹¹ CFU/mL of Corynebacterium strain(s), preferably 10³ CFU/mL to 10¹¹ CFU/mL of each Corynebacterium strain.

22. The lyophilized formulation according to claim 19, wherein
the lyophilized formulation comprises a Corynebacterium culture comprising four Corynebacterium strains, which are Corynebacterium accolens SU004, Corynebacterium tuberculostearicum SU005, Corynebacterium accolens SU006, and Corynebacterium propinquum SU010; and
the cryoprotectant comprises kappa carrageenan or xanthan.


SEQUENCE LISTING (8 sequences)

SEQ ID NO: 1 - DNA, 24 bp, synthetic construct
  source 1..24: mol_type = other DNA; note = ACE2 forward primer
gggatcagagatcggaagaagaaa

SEQ ID NO: 2 - DNA, 23 bp, synthetic construct
  source 1..23: mol_type = other DNA; note = ACE2 reverse primer
aggaggtctgaacatcatcagtg

SEQ ID NO: 3 - DNA, 21 bp, synthetic construct
  source 1..21: mol_type = other DNA; note = TMPRSS2 forward primer
aatcggtgtgttcgcctctac

SEQ ID NO: 4 - DNA, 21 bp, synthetic construct
  source 1..21: mol_type = other DNA; note = TMPRSS2 reverse primer
cgtagttctcgttccagtcgt

SEQ ID NO: 5 - DNA, 20 bp, synthetic construct
  source 1..20: mol_type = other DNA; note = Cathepsin L forward primer
ctggtggttggctacggatt

SEQ ID NO: 6 - DNA, 20 bp, synthetic construct
  source 1..20: mol_type = other DNA; note = Cathepsin L reverse primer
ctccggtctttggccatctt

SEQ ID NO: 7 - DNA, 22 bp, synthetic construct
  source 1..22: mol_type = other DNA; note = GAPDH forward primer
ctactggcgctggcaaggctgt

SEQ ID NO: 8 - DNA, 24 bp, synthetic construct
  source 1..24: mol_type = other DNA; note = GAPDH reverse primer
gccatgaggtccaccaccctgctg